(19) United States
(12) Patent Application Publication
(10) Pub. No.: US 2021/0094899 A1
(43) Pub. Date: Apr. 1, 2021

(54) ACRYLIC ACID ANALOGS

(71) Applicant: Recurium IP Holdings, LLC, San Diego, CA (US)

(72) Inventors: Peter Qinhua Huang, San Diego, CA (US); Deborah Helen Slee, Encinitas, CA (US); Kevin Duane Bunker, Escondido, CA (US); Sayee Gajanan Hegde, San Diego, CA (US); Chad Daniel Hopkins, San Diego, CA (US)

(21) Appl. No.: 16/634,366

(22) PCT Filed: Jul. 26, 2018
(86) PCT No.: PCT/US2018/043935

§ 371 Date: Jan. 27, 2020

Related U.S. Application Data
(60) Provisional application No. 62/538,486, filed on Jul. 28, 2017.

(51) Int. Cl. C07C 59/56; C07C 255/57; A61P 35/00

(52) U.S. Cl. CPC C07C 59/56; A61P 35/00; C07C 255/57

(57) ABSTRACT

Compounds of Formula (I) are provided herein. Such compounds, as well as pharmaceutically acceptable salts and compositions thereof, are useful for treating diseases or conditions, including conditions characterized by excessive cellular proliferation, such as breast cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 62/538,486, filed Jul. 28, 2017.

### FIELD

The present application relates to compounds that are estrogen receptor modulators and/or degraders and methods of using them to treat conditions characterized by excessive cellular proliferation, such as cancer.

### Description

Many cancer cells express estrogen receptors (ERs) and have growth characteristics that are modulated by estrogen, for example, breast cancer cells. ERs belong to the nuclear hormone receptor superfamily and can activate transcription of genes. In both females and males, estrogens play an important role in the regulation of a number of physiological processes. Humans are known to possess two different ER subtypes: ERα and ERβ. Each subtype has a distinct tissue distribution and with different biological roles. For example, ERα has high presence in endometrium, breast cancer cells, ovarian stroma cells and in the hypothalamus. The expression of ERβ has been documented in kidney, brain, bone, heart, lungs, intestinal mucosa, prostate, bladder, ovary, testis and endothelial cells.

### SUMMARY

Some embodiments provide a compound of Formula (I), or a pharmaceutically acceptable salt thereof.

Some embodiments disclosed herein relate to a pharmaceutical composition that can include an effective amount of one or more of compounds of Formula (I), or a pharmaceutically acceptable salt thereof, and a pharmaceutically acceptable carrier, diluent, excipient or combination thereof.

Some embodiments disclosed herein relate to a method of treatment that can include identifying a subject that is in need of treatment for a disease or condition that is ER alpha dependent, and/or ER alpha mediated; and administering to said subject an effective amount of a compound of Formula (I), or a pharmaceutically acceptable salt thereof, or a pharmaceutical composition that can include a compound of Formula (I), or a pharmaceutically acceptable salt thereof. Other embodiments disclosed herein relate to the use of a compound of Formula (I), or a pharmaceutically acceptable salt thereof, or a pharmaceutical composition that can include a compound of Formula (I), or a pharmaceutically acceptable salt thereof, in the preparation of a medicament for the treatment of a disease or condition that is ER alpha dependent, and/or ER alpha mediated. Still other embodiments disclosed herein relate to a compound of Formula (I), or a pharmaceutically acceptable salt thereof, or a pharmaceutical composition that can include a compound of Formula (I), or a pharmaceutically acceptable salt thereof, for use in the treatment of a disease or condition that is ER alpha dependent, and/or ER alpha mediated.

Some embodiments disclosed herein relate a method of inhibiting the growth of a cell, that can include identifying a cell having an ER alpha that mediates a growth characteristic of the cell; and contacting the cell with an effective amount of the compound of Formula (I), or a pharmaceutically acceptable salt thereof. Other embodiments disclosed herein relate to the use of a compound of Formula (I), or a pharmaceutically acceptable salt thereof, or a pharmaceutical composition that can include a compound of Formula (I), or a pharmaceutically acceptable salt thereof, in the preparation of a medicament for inhibiting the growth of a cell, that has an ER alpha that mediates a growth characteristic of the cell. Still other embodiments disclosed herein relate to a compound of Formula (I), or a pharmaceutically acceptable salt thereof, or a pharmaceutical composition that can include a compound of Formula (I), or a pharmaceutically acceptable salt thereof, for use in inhibiting the growth of a cell that has an ER alpha that mediates a growth characteristic of the cell.

### DETAILED DESCRIPTION

A number of breast cancer drug therapies have been developed that target ERs. In many cases the drugs are selective ER modulators (SERMs) that have agonistic and/or antagonistic effects on ERs. For example, fulvestrant is a drug that is used for the treatment of metastatic breast cancer. It has antagonistic effects on ER alpha and is considered a selective ER alpha degrader (SERD). Fulvestrant has the following chemical structure:

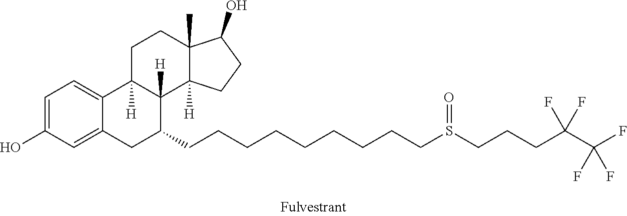

Other SERDs include, for example, elacestrant (RAD1901), brilanestrant (GDC-0810) and AZD9496. See Garner, et al., Anti-Cancer Drugs 26(9), 948-956 (2015), De Savi, et al., J. Med. Chem. 58, 8128-8140 (2015) and Lai, et al., J. Med. Chem. 58, 4888-4904 (2015), respectively.

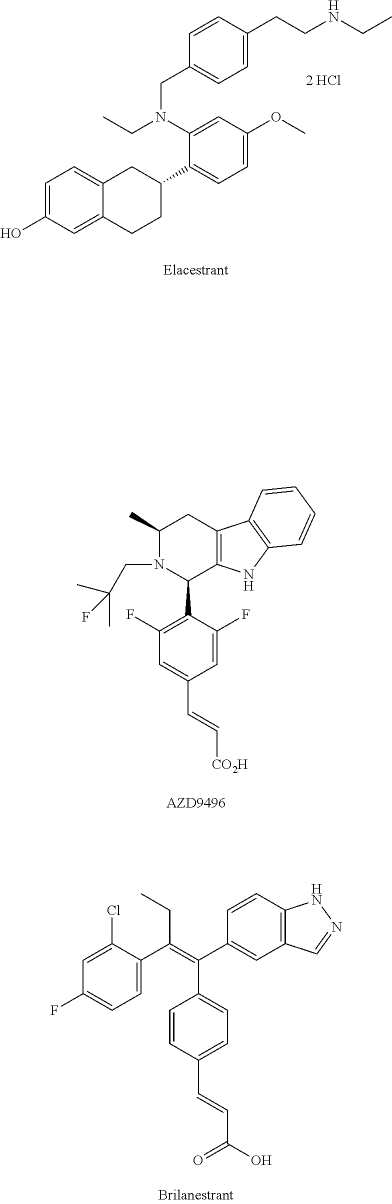

Structured similarly to all nuclear receptors, ERα includes six functional domains and is classified as a ligand-dependent transcription factor. After its association with 17β estradiol (E2), the complex binds to genomic sequences, named Estrogen Receptor Elements (EREs) to modulate the transcription of target genes. A large number of structurally distinct compounds have been shown to bind to ERs. These compounds can be divided into 2 classes depending on their functional effects. Selective estrogen receptor modulators (SERMs) such as tamoxifen act as both receptor agonists and antagonists. A second group, fulvestrant being an example, are full antagonists. Fulvestrant is currently the only SERD approved for clinical use, yet despite its mechanistic properties, the pharmacological properties of the drug have limited its efficacy due to its poor absorption and the current limitation of a 500 mg monthly dose which results in less than 50% turnover of the receptor in patient samples compared to the complete down-regulation of the receptor seen in in vitro breast cell line experiments. See, e.g., Wardell, et al., Biochem. Pharm., Vol. 82, pp. 122-130 (2011).

The clinical efficacy of fulvestrant is also limited as it must be dosed via intramuscular injection. A number of orally dosed SERDs are currently in clinical development, e.g., brilanestrant, elacestrant, AZD9496, LSZ102, H3B-6545, SAR439859, G1T48, and SRN-927, but at this time it appears that no oral SERD has been approved for the treatment of breast cancer in the United States. See De Savi, C. et al. J. Med. Chem. 58, 8128-8140 (2015). Thus, there remains a long-felt need for well tolerated orally dosed SERDs and/or SERMs that are useful in the study and the treatment of proliferative disorders, such as breast cancer, that have growth characteristics that are modulated by estrogen.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, “growth characteristics,” refer to aspects of cell growth and development, including, but not limited to, cellular proliferation potential, cellular division rate, cell adhesion, contact inhibition, cell mobility, cellular response in the presence/absence of growth factors, cell cycle regulation, expression and function of cell surface receptors and programmed cell death.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group and a di-substituted amine group.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

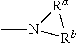

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of monocyclic cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5] decane.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, “lower alkylene groups” are straight-chained —CH₂-tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group and/or by substituting both hydrogens on the same carbon with a cycloalkyl group (e.g.,

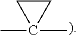

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})-” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)═N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The term “amino” as used herein refers to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amino groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), 13 N(ethyl)(methyl) and the like.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like; the term ‘comprising’ as used herein is synonymous with ‘including,’ containing,' or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ preferred,“desired,” or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to a compound of Formula (I), or a pharmaceutically acceptable salt thereof, having the structure:

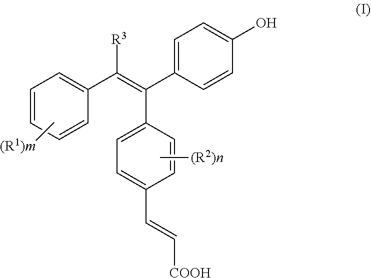

wherein: one or more of R¹ can be independently selected from halogen, —CN, an optionally substituted C₁-C₆ alkyl, an unsubstituted C₁-C₆ haloalkyl and an optionally substituted C₃-C₆ cycloalkyl; one or more of R² can be independently selected from halogen, —CN, an optionally substituted C₁-C₆ alkyl, an unsubstituted C₁-C₆ haloalkyl and an optionally substituted C₃-C₆ cycloalkyl; R³ is selected from the group consisting of an optionally substituted C₁-C₆ alkyl, an unsubstituted C₁-C₆ haloalkyl and an optionally substituted C₃-C₆ cycloalkyl; m can be 0, 1, 2, 3, 4 or 5; n can be 0, 1, 2, 3 or 4; and when m is 0, then n is 1, 2, 3 or 4.

In some embodiments, one or more of R¹ can be independently halogen. In some embodiments, one or more of R¹ can be fluoro. In other embodiments, one or more of R¹ can be chloro. In some embodiments, each R¹ can be fluoro. In other embodiments, each R¹ can be chloro.

In some embodiments, one or more of R¹ can be —CN. In other embodiments, one or more of R¹ can be independently an optionally substituted C₁-C₆ alkyl. In some embodiments, one or more of R¹ can be independently a substituted C₁-C₆ alkyl. In some embodiments, one or more of R¹ can be independently an unsubstituted C₁-C₆ alkyl. Examples of C₁-C₆ alkyl groups include methyl, ethyl, n-propyl, iso-propyl, n-butyl, iso-butyl, tert-butyl, pentyl (straight-chained or branched) and hexyl (straight-chained or branched). In some embodiments, one or more of R¹ can be methyl. In still other embodiments, one or more of R¹ can be independently an unsubstituted C₁-C₆ haloalkyl, such —CF₃, —CCl₃, —CHF, —CHCl₂, —CHF, —CH₂Cl, —CH₂CF₃, —CF₂CHF₂, —CF₂CF₃and —CF₂Cl. In some embodiments, one or more of R¹ can be independently an optionally substituted C₃-C₆ cycloalkyl. In some embodiments, one or more of R¹ can be independently a substituted C₃-C₆ cycloalkyl. In some embodiments, one or more of R¹ can be independently an unsubstituted C₃-C₆ cycloalkyl. When R¹ is an optionally substituted C₃-C₆ cycloalkyl, the C₃-C₆ cycloalkyl can be monocyclic or bridged bicyclic. Examples of C₃-C₆ cycloalkyl include, but are not limited to, cyclopropyl, cyclobutyl, cyclopentyl, cyclohexyl and bicyclo[1.1.1]pentyl, wherein each of the aforementioned groups can be optionally substituted.

In some embodiments, m can be 0. In other embodiments, m can be 1. In still other embodiments, m can be 2. In yet still embodiments, m can be 3. In some embodiments, m can be 4. In some embodiments, m can be 5. When m is greater than 1, one or more of the R¹ groups can be the same and/or one or more of the R¹ groups can be different. For example, when m is 2, one R¹ can be fluoro and one R¹ can be chloro. As another example, when m is 2, one R¹ can be fluoro and one R¹ can be an optionally substituted C₁-C₆ alkyl, such as an unsubstituted methyl.

In some embodiments, one or more of R² can be independently halogen. In some embodiments, one or more of R² can be fluoro. In other embodiments, one or more of R² can be chloro. In some embodiments, each R² can be fluoro. In other embodiments, each R² can be chloro.

In some embodiments, one or more of R² can be —CN. In other embodiments, one or more of R² can be independently an optionally substituted C₁-C₆ alkyl. In some embodiments, one or more of R² can be independently a substituted C₁-C₆ alkyl. In some embodiments, one or more of R² can be independently an unsubstituted C₁-C₆ alkyl. Examples of C₁-C₆ alkyl groups include methyl, ethyl, n-propyl, iso-propyl, n-butyl, iso-butyl, tert-butyl, pentyl (straight-chained or branched) and hexyl (straight-chained or branched). In some embodiments, one or more of R² can be methyl. In still other embodiments, one or more of R² can be independently an unsubstituted C₁-C₆ haloalkyl. For example, R² can be —CF₃, —CCl₃, —CHF₂, —CHCl₂, —CH₂F, —CH₂Cl, —CH₂CF₃, —CF₂CHF₂, —CF₂CF₃and —CF₂Cl. In some embodiments, one or more of R² can be independently an optionally substituted C₃-C₆ cycloalkyl.

In some embodiments, n can be 0. In other embodiments, n can be 1. In still other embodiments, n can be 2. In yet still other embodiments, n can be 3. In some embodiments, n can be 4. When n is greater than 1, one or more of the R² groups can be the same and/or one or more of the R² groups can be different. For example, when n is greater than 1, each R² group can be fluoro.

In some embodiments, R³ can be an optionally substituted C₁-C₆ alkyl. In some embodiments, R³ can be a substituted C₁-C₆ alkyl. In some embodiments, R³ can be an unsubstituted C₁-C₆ alkyl. Examples of C₁-C₆ alkyl groups include methyl, ethyl, n-propyl, iso-propyl, n-butyl, iso-butyl, tert-butyl, pentyl (straight-chained or branched) and hexyl (straight-chained or branched). In some embodiments R³ can be ethyl. In still other embodiments, R³ can be an unsubstituted C₁-C₆ haloalkyl, such as —CF₃, —CCl₃, —CHF₂, —CHCl₂, —CH₂F, —CH₂Cl, —CH₂CF₃, —CF₂CHF₂, —CF₂CF₃and —CF₂Cl. In some embodiments, R³ can be an optionally substituted C₃-C₆ cycloalkyl. In some embodiments, R³ can be a substituted C₃-C₆ cycloalkyl. In some embodiments, R³ can be an unsubstituted C₃-C₆ cycloalkyl. When R³ is an optionally substituted C₃-C₆ cycloalkyl, the C₃-C₆ cycloalkyl can be monocyclic or bridged bicyclic. Examples of C₃-C₆ cycloalkyl include, but are not limited to, cyclopropyl, cyclobutyl, cyclopentyl, cyclohexyl and bicyclo[1.1.1]pentyl, wherein each of the aforementioned groups can be optionally substituted.

In some embodiments, m can be 1, 2 or 3; and one or more of R¹ can be independently selected from halogen (such as chloro or fluoro), an unsubstituted C₁-C₆ alkyl and an unsubstituted C₁-C₆ haloalkyl. In other embodiments, m can be 1 or 2; and one or more of R¹ can be independently halogen or an unsubstituted C₁-C₆ alkyl. In some embodiments, m can be 1 or 2; and each R¹ can be fluoro. In other embodiments, m can be 1 or 2; and each R¹ can be chloro. In still other embodiments, m can be 1 or 2; and each R¹ can be methyl. In some of the embodiments of this paragraph, R³ can be an unsubstituted C₁-C₆ alkyl, such as ethyl.

In some embodiments, n can be 0, 1 or 2; and one or more of R² can be independently selected from halogen (for example, fluoro or chloro), an unsubstituted C₁-C₆ alkyl and an unsubstituted C₁-C₆ haloalkyl. In other embodiments, n can be 1 or 2; and one or more of R² can be halogen or an unsubstituted C₁-C₆ alkyl. In some embodiments, n can be 1 or 2; and each R² can be fluoro. In other embodiments, n can be 1 or 2; and each R² can be chloro. In still other embodiments, n can be 1 or 2; and each R² can be methyl. In some embodiments, m can be 2; n can be 0; one R¹ can be chloro; and the other R¹ can be fluoro. In other embodiments, m can be 2; n can be 0; one R¹ can be fluoro; and the other R¹ can be unsubstituted C₁-C₆ alkyl, such as methyl. In still other embodiments, m can be 1; n can be 0; and R¹ can be chloro. In yet still other embodiments, m can be 2; n can be 2; one R¹ can be chloro; the other R¹ can be fluoro; and each R² can be fluoro. In some embodiments, m can be 2; n can be 2; one R¹ can be fluoro; the other R¹ can be unsubstituted C₁-C₆ alkyl, such as methyl; and each R² can be fluoro. In other embodiments, m can be 1; n can be 2; R¹ can be chloro; and each R² can be fluoro. In some of the embodiments of this paragraph, R³ can be an unsubstituted C₁-C₆ alkyl, such as ethyl. Further embodiments are provided below in Tables 1 and 2. In some of the embodiments of Tables 1 and 2, R³ can be an unsubstituted C₁-C₆ alkyl, such as ethyl.

Some embodiments of m and n of a compound of Formula (I), or a pharmaceutically acceptable salt thereof, are provided in Table 1. For example, the first entry in Table 1 is “A” and corresponds to a compound of Formula (I), or a pharmaceutically acceptable salt thereof, wherein m is 0 and n is 1.

TABLE 1

| m | n |
|---|---|
| 0 | 1 |
| 0 | 2 |
| 0 | 3 |
| 0 | 4 |
| 1 | 0 |
| 1 | 1 |
| 1 | 2 |
| 1 | 3 |
| 1 | 4 |
| 2 | 0 |
| 2 | 1 |
| 2 | 2 |
| 2 | 3 |
| 2 | 4 |
| 3 | 0 |
| 3 | 1 |
| 3 | 2 |
| 3 | 3 |
| 3 | 4 |
| 4 | 0 |
| 4 | 1 |
| 4 | 2 |
| 4 | 3 |
| 4 | 4 |
| 5 | 0 |
| 5 | 1 |
| 5 | 2 |
| 5 | 3 |
| 5 | 4 |
| — | — |

TABLE 2

| m | n | R¹ | R² |
|---|---|---|---|
| 0 | 1 | — | halogen |
| 0 | 1 | — | an optionally substituted C₁-C₆ alkyl |
| 0 | 1 | — | an unsubstituted C₁-C₆ haloalkyl |
| 0 | 2 | — | an optionally substituted C₁-C₆ alkyl |
| | | | & an optionally substituted C₁-C₆ |
| | | | alkyl |
| 0 | 2 | — | an unsubstituted C₁-C₆ haloalkyl & an |
| | | | unsubstituted C₁-C₆ haloalkyl |
| 0 | 2 | — | halogen & an optionally substituted |
| | | | C₁-C₆ alkyl |
| 0 | 2 | — | halogen & an unsubstituted C₁-C₆ |
| | | | haloalkyl |
| 0 | 2 | — | halogen & halogen |
| 0 | 2 | — | an optionally substituted C₁-C₆ alkyl |
| | | | & an optionally substituted C₁-C₆ |
| | | | alkyl |
| 0 | 2 | — | an optionally substituted C₁-C₆ alkyl |
| | | | & an unsubstituted C₁-C₆ haloalkyl |
| 0 | 2 | — | an unsubstituted C₁-C₆ haloalkyl & an |
| | | | unsubstituted C₁-C₆ haloalkyl |
| 0 | 3 | — | halogen, an optionally substituted C₁-C₆ |
| | | | alkyl & an unsubstituted C₁-C₆ |
| | | | haloalkyl |
| 0 | 3 | — | halogen, halogen & halogen |
| 0 | 3 | — | halogen, halogen & an optionally |
| | | | substituted C₁-C₆ alkyl |
| 0 | 3 | — | halogen, halogen & an unsubstituted |
| | | | C₁-C₆ haloalkyl |
| 0 | 3 | — | halogen, an optionally substituted C₁-C₆ |
| | | | alkyl & an optionally substituted |
| | | | C₁-C₆ alkyl |
| 0 | 3 | — | halogen, an unsubstituted C₁-C₆ |
| | | | haloalkyl & an unsubstituted C₁-C₆ |
| | | | haloalkyl |
| 0 | 3 | — | an optionally substituted C₁-C₆ alkyl, |
| | | | an optionally substituted C₁-C₆ alkyl |
| | | | & an unsubstituted C₁-C₆ haloalkyl |
| 0 | 3 | — | an optionally substituted C₁-C₆ alkyl, |
| | | | an unsubstituted C₁-C₆ haloalkyl & an |
| | | | unsubstituted C₁-C₆ haloalkyl |
| 1 | 1 | halogen | halogen |
| 1 | 1 | halogen | an optionally substituted C₁-C₆ alkyl |
| 1 | 1 | halogen | an unsubstituted C₁-C₆ haloalkyl |
| 1 | 1 | —CN | halogen |
| 1 | 1 | —CN | an optionally substituted C₁-C₆ alkyl |
| 1 | 1 | —CN | an unsubstituted C₁-C₆ haloalkyl |
| 1 | 1 | an optionally substituted C₁-C₆ alkyl | halogen |
| 1 | 1 | an optionally substituted C₁-C₆ alkyl | an optionally substituted C₁-C₆ alkyl |
| 1 | 1 | an optionally substituted C₁-C₆ alkyl | an unsubstituted C₁-C₆ haloalkyl |
| 1 | 1 | an unsubstituted C₁-C₆ haloalkyl | halogen |
| 1 | 1 | an unsubstituted C₁-C₆ haloalkyl | an optionally substituted C₁-C₆ alkyl |
| 1 | 1 | an unsubstituted C₁-C₆ haloalkyl | an unsubstituted C₁-C₆ haloalkyl |
| 1 | 2 | halogen | halogen & halogen |
| 1 | 2 | halogen | halogen & an optionally substituted |
| | | | C₁-C₆ alkyl |
| 1 | 2 | halogen | halogen & an unsubstituted C₁-C₆ |
| | | | haloalkyl |
| 1 | 2 | halogen | an optionally substituted C₁-C₆ alkyl |
| | | | & an optionally substituted C₁-C₆ |
| | | | alkyl |
| 1 | 2 | halogen | an optionally substituted C₁-C₆ alkyl |
| | | | & an unsubstituted C₁-C₆ haloalkyl |
| 1 | 2 | halogen | an unsubstituted C₁-C₆ haloalkyl & an |
| | | | unsubstituted C₁-C₆ haloalkyl |
| 1 | 2 | —CN | halogen & halogen |
| 1 | 2 | —CN | halogen & an optionally substituted |
| | | | C₁-C₆ alkyl |
| 1 | 2 | —CN | halogen & an unsubstituted C₁-C₆ |
| | | | haloalkyl |
| 1 | 2 | —CN | an optionally substituted C₁-C₆ alkyl |
| | | | & an optionally substituted C₁-C₆ |
| | | | alkyl |
| 1 | 2 | —CN | an optionally substituted C₁-C₆ alkyl |
| | | | & an unsubstituted C₁-C₆ haloalkyl |
| 1 | 2 | —CN | an unsubstituted C₁-C₆ haloalkyl & an |
| | | | unsubstituted C₁-C₆ haloalkyl |
| 1 | 2 | an optionally substituted C₁-C₆ alkyl | halogen & halogen |
| 1 | 2 | an optionally substituted C₁-C₆ alkyl | halogen & an optionally substituted |
| | | | C₁-C₆ alkyl |
| 1 | 2 | an optionally substituted C₁-C₆ alkyl | halogen & an unsubstituted C₁-C₆ |
| | | | haloalkyl |
| 1 | 2 | an optionally substituted C₁-C₆ alkyl | an optionally substituted C₁-C₆ alkyl |
| | | | & an optionally substituted C₁-C₆ |
| | | | alkyl |
| 1 | 2 | an optionally substituted C₁-C₆ alkyl | an optionally substituted C₁-C₆ alkyl |
| | | | & an unsubstituted C₁-C₆ haloalkyl |
| 1 | 2 | an optionally substituted C₁-C₆ alkyl | an unsubstituted C₁-C₆ haloalkyl & an |
| | | | unsubstituted C₁-C₆ haloalkyl |
| 1 | 2 | an unsubstituted C₁-C₆ haloalkyl | halogen & halogen |
| 1 | 2 | an unsubstituted C₁-C₆ haloalkyl | halogen & an optionally substituted |
| | | | C₁-C₆ alkyl |
| 1 | 2 | an unsubstituted C₁-C₆ haloalkyl | halogen & an unsubstituted C₁-C₆ |
| | | | haloalkyl |
| 1 | 2 | an unsubstituted C₁-C₆ haloalkyl | an optionally substituted C₁-C₆ alkyl |
| | | | & an optionally substituted C₁-C₆ |
| | | | alkyl |
| 1 | 2 | an unsubstituted C₁-C₆ haloalkyl | an optionally substituted C₁-C₆ alkyl |
| | | | & an unsubstituted C₁-C₆ haloalkyl |
| 1 | 2 | an unsubstituted C₁-C₆ haloalkyl | an unsubstituted C₁-C₆ haloalkyl & an |
| | | | unsubstituted C₁-C₆ haloalkyl |
| 1 | 3 | halogen | halogen, an optionally substituted C₁-C₆ |
| | | | alkyl & an unsubstituted C₁-C₆ |
| | | | haloalkyl |
| 1 | 3 | halogen | halogen, halogen & halogen |
| 1 | 3 | halogen | halogen, halogen & an optionally |
| | | | substituted C₁-C₆ alkyl |
| 1 | 3 | halogen | halogen, halogen & an unsubstituted |
| | | | C₁-C₆ haloalkyl |
| 1 | 3 | halogen | halogen, an optionally substituted C₁-C₆ |
| | | | alkyl & an optionally substituted |
| | | | C₁-C₆ alkyl |
| 1 | 3 | halogen | halogen, an unsubstituted C₁-C₆ |
| | | | haloalkyl & an unsubstituted C₁-C₆ |
| | | | haloalkyl |
| 1 | 3 | halogen | an optionally substituted C₁-C₆ alkyl, |
| | | | an optionally substituted C₁-C₆ alkyl |
| | | | & an unsubstituted C₁-C₆ haloalkyl |
| 1 | 3 | halogen | an optionally substituted C₁-C₆ alkyl, |
| | | | an unsubstituted C₁-C₆ haloalkyl & an |
| | | | unsubstituted C₁-C₆ haloalkyl |
| 1 | 3 | —CN | halogen, an optionally substituted C₁-C₆ |
| | | | alkyl & an unsubstituted C₁-C₆ |
| | | | haloalkyl |
| 1 | 3 | —CN | halogen, halogen & halogen |
| 1 | 3 | —CN | halogen, halogen & an optionally |
| | | | substituted C₁-C₆ alkyl |
| 1 | 3 | —CN | halogen, halogen & an unsubstituted |
| | | | C₁-C₆ haloalkyl |
| 1 | 3 | —CN | halogen, an optionally substituted C₁-C₆ |
| | | | alkyl & an optionally substituted |
| | | | C₁-C₆ alkyl |
| 1 | 3 | —CN | halogen, an unsubstituted C₁-C₆ |
| | | | haloalkyl & an unsubstituted C₁-C₆ |
| | | | haloalkyl |
| 1 | 3 | —CN | an optionally substituted C₁-C₆ alkyl, |
| | | | an optionally substituted C₁-C₆ alkyl |
| | | | & an unsubstituted C₁-C₆ haloalkyl |
| 1 | 3 | —CN | an optionally substituted C₁-C₆ alkyl, |
| | | | an unsubstituted C₁-C₆ haloalkyl & an |
| | | | unsubstituted C₁-C₆ haloalkyl |
| 1 | 3 | an optionally substituted C₁-C₆ alkyl | halogen, an optionally substituted C₁-C₆ |
| | | | alkyl & an unsubstituted C₁-C₆ |
| | | | haloalkyl |
| 1 | 3 | an optionally substituted C₁-C₆ alkyl | halogen, halogen & halogen |
| 1 | 3 | an optionally substituted C₁-C₆ alkyl | halogen, halogen & an optionally |
| | | | substituted C₁-C₆ alkyl |
| 1 | 3 | an optionally substituted C₁-C₆ alkyl | halogen, halogen & an unsubstituted |
| | | | C₁-C₆ haloalkyl |
| 1 | 3 | an optionally substituted C₁-C₆ alkyl | halogen, an optionally substituted C₁-C₆ |
| | | | alkyl & an optionally substituted |
| | | | C₁-C₆ alkyl |
| 1 | 3 | an optionally substituted C₁-C₆ alkyl | halogen, an unsubstituted C₁-C₆ |
| | | | haloalkyl & an unsubstituted C₁-C₆ |
| | | | haloalkyl |
| 1 | 3 | an optionally substituted C₁-C₆ alkyl | an optionally substituted C₁-C₆ alkyl, |
| | | | an optionally substituted C₁-C₆ alkyl |
| | | | & an unsubstituted C₁-C₆ haloalkyl |
| 1 | 3 | an optionally substituted C₁-C₆ alkyl | an optionally substituted C₁-C₆ alkyl, |
| | | | an unsubstituted C₁-C₆ haloalkyl & an |
| | | | unsubstituted C₁-C₆ haloalkyl |
| 1 | 3 | an unsubstituted C₁-C₆ haloalkyl | halogen, an optionally substituted C₁-C₆ |
| | | | alkyl & an unsubstituted C₁-C₆ |
| | | | haloalkyl |
| 1 | 3 | an unsubstituted C₁-C₆ haloalkyl | halogen, halogen & halogen |
| 1 | 3 | an unsubstituted C₁-C₆ haloalkyl | halogen, halogen & an optionally |
| | | | substituted C₁-C₆ alkyl |
| 1 | 3 | an unsubstituted C₁-C₆ haloalkyl | halogen, halogen & an unsubstituted |
| | | | C₁-C₆ haloalkyl |
| 1 | 3 | an unsubstituted C₁-C₆ haloalkyl | halogen, an optionally substituted C₁-C₆ |
| | | | alkyl & an optionally substituted |
| | | | C₁-C₆ alkyl |
| 1 | 3 | an unsubstituted C₁-C₆ haloalkyl | halogen, an unsubstituted C₁-C₆ |
| | | | haloalkyl & an unsubstituted C₁-C₆ |
| | | | haloalkyl |
| 1 | 3 | an unsubstituted C₁-C₆ haloalkyl | an optionally substituted C₁-C₆ alkyl, |
| | | | an optionally substituted C₁-C₆ alkyl |
| | | | & an unsubstituted C₁-C₆ haloalkyl |
| 1 | 3 | an unsubstituted C₁-C₆ haloalkyl | an optionally substituted C₁-C₆ alkyl, |
| | | | an unsubstituted C₁-C₆ haloalkyl & an |
| | | | unsubstituted C₁-C₆ haloalkyl |
| 2 | 0 | halogen & halogen | — |
| 2 | 0 | halogen & an optionally substituted | — |
| | | C₁-C₆ alkyl | |
| 2 | 0 | halogen & an unsubstituted C₁-C₆ | — |
| | | haloalkyl | |
| 2 | 0 | an optionally substituted C₁-C₆ alkyl | — |
| | | & an optionally substituted C₁-C₆ | |
| | | alkyl | |
| 2 | 0 | an optionally substituted C₁-C₆ alkyl | — |
| | | & an unsubstituted C₁-C₆ haloalkyl | |
| 2 | 0 | an unsubstituted C₁-C₆ haloalkyl & | — |
| | | an unsubstituted C₁-C₆ haloalkyl | |
| 2 | 0 | halogen & CN | — |
| 2 | 0 | an optionally substituted C₁-C₆ alky | — |
| | | & CN | |
| 2 | 0 | an unsubstituted C₁-C₆ haloalkyl | — |
| | | &CN | |
| 2 | 2 | halogen & halogen | halogen & halogen |
| 2 | 2 | halogen & halogen | halogen & an optionally substituted |
| | | | C₁-C₆ alkyl |
| 2 | 2 | halogen & halogen | halogen & an unsubstituted C₁-C₆ |
| | | | haloalkyl |
| 2 | 2 | halogen & halogen | an optionally substituted C₁-C₆ alkyl |
| | | | & an optionally substituted C₁-C₆ |
| | | | alkyl |
| 2 | 2 | halogen & halogen | an optionally substituted C₁-C₆ alkyl |
| | | | & an unsubstituted C₁-C₆ haloalkyl |
| 2 | 2 | halogen & halogen | an unsubstituted C₁-C₆ haloalkyl & an |
| | | | unsubstituted C₁-C₆ haloalkyl |
| 2 | 2 | halogen & an optionally substituted | halogen & halogen |
| | | C₁-C₆ alkyl | |
| 2 | 2 | halogen & an optionally substituted | halogen & an optionally substituted |
| | | C₁-C₆ alkyl | C₁-C₆ alkyl |
| 2 | 2 | halogen & an optionally substituted | halogen & an unsubstituted C₁-C₆ |
| | | C₁-C₆ alkyl | haloalkyl |
| 2 | 2 | halogen & an optionally substituted | an optionally substituted C₁-C₆ alkyl |
| | | C₁-C₆ alkyl | & an optionally substituted C₁-C₆ |
| | | | alkyl |
| 2 | 2 | halogen & an optionally substituted | an optionally substituted C₁-C₆ alkyl |
| | | C₁-C₆ alkyl | & an unsubstituted C₁-C₆ haloalkyl |
| 2 | 2 | halogen & an optionally substituted | an unsubstituted C₁-C₆ haloalkyl & an |
| | | C₁-C₆ alkyl | unsubstituted C₁-C₆ haloalkyl |
| 2 | 2 | halogen & an unsubstituted C₁-C₆ | halogen & halogen |
| | | haloalkyl | |
| 2 | 2 | halogen & an unsubstituted C₁-C₆ | halogen & an optionally substituted |
| | | haloalkyl | C₁-C₆ alkyl |
| 2 | 2 | halogen & an unsubstituted C₁-C₆ | halogen & an unsubstituted C₁-C₆ |
| | | haloalkyl | haloalkyl |
| 2 | 2 | halogen & an unsubstituted C₁-C₆ | an optionally substituted C₁-C₆ alkyl |
| | | haloalkyl | & an optionally substituted C₁-C₆ |
| | | | alkyl |
| 2 | 2 | halogen & an unsubstituted C₁-C₆ | an optionally substituted C₁-C₆ alkyl |
| | | haloalkyl | & an unsubstituted C₁-C₆ haloalkyl |
| 2 | 2 | halogen & an unsubstituted C₁-C₆ | an unsubstituted C₁-C₆ haloalkyl & an |
| | | haloalkyl | unsubstituted C₁-C₆ haloalkyl |
| 2 | 2 | an optionally substituted C₁-C₆ alkyl | halogen & halogen |
| | | & an optionally substituted C₁-C₆ | |
| | | alkyl | |
| 2 | 2 | an optionally substituted C₁-C₆ alkyl | halogen & an optionally substituted |
| | | & an optionally substituted C₁-C₆ | C₁-C₆ alkyl |
| | | alkyl | |
| 2 | 2 | an optionally substituted C₁-C₆ alkyl | halogen & an unsubstituted C₁-C₆ |
| | | & an optionally substituted C₁-C₆ | haloalkyl |
| | | alkyl | |
| 2 | 2 | an optionally substituted C₁-C₆ alkyl | an optionally substituted C₁-C₆ alkyl |
| | | & an optionally substituted C₁-C₆ | & an optionally substituted C₁-C₆ |
| | | alkyl | alkyl |
| 2 | 2 | an optionally substituted C₁-C₆ alkyl | an optionally substituted C₁-C₆ alkyl |
| | | & an optionally substituted C₁-C₆ | & an unsubstituted C₁-C₆ haloalkyl |
| | | alkyl | |
| 2 | 2 | an optionally substituted C₁-C₆ alkyl | an unsubstituted C₁-C₆ haloalkyl & an |
| | | & an optionally substituted C₁-C₆ | unsubstituted C₁-C₆ haloalkyl |
| | | alkyl | |
| 2 | 2 | an optionally substituted C₁-C₆ alkyl | halogen & halogen |
| | | & an unsubstituted C₁-C₆ haloalkyl | |
| 2 | 2 | an optionally substituted C₁-C₆ alkyl | halogen & an optionally substituted |
| | | & an unsubstituted C₁-C₆ haloalkyl | C₁-C₆ alkyl |
| 2 | 2 | an optionally substituted C₁-C₆ alkyl | halogen & an unsubstituted C₁-C₆ |
| | | & an unsubstituted C₁-C₆ haloalkyl | haloalkyl |
| 2 | 2 | an optionally substituted C₁-C₆ alkyl | an optionally substituted C₁-C₆ alkyl |
| | | & an unsubstituted C₁-C₆ haloalkyl | & an optionally substituted C₁-C₆ |
| | | | alkyl |
| 2 | 2 | an optionally substituted C₁-C₆ alkyl | an optionally substituted C₁-C₆ alkyl |
| | | & an unsubstituted C₁-C₆ haloalkyl | & an unsubstituted C₁-C₆ haloalkyl |
| 2 | 2 | an optionally substituted C₁-C₆ alkyl | an unsubstituted C₁-C₆ haloalkyl & an |
| | | & an unsubstituted C₁-C₆ haloalkyl | unsubstituted C₁-C₆ haloalkyl |
| 2 | 2 | an unsubstituted C₁-C₆ haloalkyl & | halogen & halogen |
| | | an unsubstituted C₁-C₆ haloalkyl | |
| 2 | 2 | an unsubstituted C₁-C₆ haloalkyl & | halogen & an optionally substituted |
| | | an unsubstituted C₁-C₆ haloalkyl | C₁-C₆ alkyl |
| 2 | 2 | an unsubstituted C₁-C₆ haloalkyl & | halogen & an unsubstituted C₁-C₆ |
| | | an unsubstituted C₁-C₆ haloalkyl | haloalkyl |
| 2 | 2 | an unsubstituted C₁-C₆ haloalkyl & | an optionally substituted C₁-C₆ alkyl |
| | | an unsubstituted C₁-C₆ haloalkyl | & an optionally substituted C₁-C₆ |
| | | | alkyl |
| 2 | 2 | an unsubstituted C₁-C₆ haloalkyl & | an optionally substituted C₁-C₆ alkyl |
| | | an unsubstituted C₁-C₆ haloalkyl | & an unsubstituted C₁-C₆ haloalkyl |
| 2 | 2 | an unsubstituted C₁-C₆ haloalkyl & | an unsubstituted C₁-C₆ haloalkyl & an |
| | | an unsubstituted C₁-C₆ haloalkyl | unsubstituted C₁-C₆ haloalkyl |
| 2 | 2 | —CN & halogen | halogen & halogen |
| 2 | 2 | —CN & halogen | halogen & an optionally substituted |
| | | | C₁-C₆ alkyl |
| 2 | 2 | —CN & halogen | halogen & an unsubstituted C₁-C₆ |
| | | | haloalkyl |
| 2 | 2 | —CN & halogen | an optionally substituted C₁-C₆ alkyl |
| | | | & an optionally substituted C₁-C₆ |
| | | | alkyl |
| 2 | 2 | —CN & halogen | an optionally substituted C₁-C₆ alkyl |
| | | | & an unsubstituted C₁-C₆ haloalkyl |
| 2 | 2 | —CN & halogen | an unsubstituted C₁-C₆ haloalkyl & an |
| | | | unsubstituted C₁-C₆ haloalkyl |
| 2 | 2 | —CN & an optionally substituted C₁-C₆ alkyl | halogen & halogen |
| 2 | 2 | —CN & an optionally substituted C₁-C₆ alkyl | halogen & an optionally substituted |
| | | | C₁-C₆ alkyl |
| 2 | 2 | —CN & an optionally substituted C₁-C₆ alkyl | halogen & an unsubstituted C₁-C₆ |
| | | | haloalkyl |
| 2 | 2 | —CN & an optionally substituted C₁-C₆ alkyl | an optionally substituted C₁-C₆ alkyl |
| | | | & an optionally substituted C₁-C₆ |
| | | | alkyl |
| 2 | 2 | —CN & an optionally substituted C₁-C₆ alkyl | an optionally substituted C₁-C₆ alkyl |
| | | | & an unsubstituted C₁-C₆ haloalkyl |
| 2 | 2 | —CN & an optionally substituted C₁-C₆ alkyl | an unsubstituted C₁-C₆ haloalkyl & an |
| | | | unsubstituted C₁-C₆ haloalkyl |
| 2 | 2 | —CN & an unsubstituted C₁-C₆ | halogen & halogen |
| | | haloalkyl | |
| 2 | 2 | —CN & an unsubstituted C₁-C₆ | halogen & an optionally substituted |
| | | haloalkyl | C₁-C₆ alkyl |
| 2 | 2 | —CN & an unsubstituted C₁-C₆ | halogen & an unsubstituted C₁-C₆ |
| | | haloalkyl | haloalkyl |
| 2 | 2 | —CN & an unsubstituted C₁-C₆ | an unsubstituted C₁-C₆ haloalkyl & an |
| | | haloalkyl | unsubstituted C₁-C₆ haloalkyl |
| 2 | 3 | halogen & halogen | halogen, an optionally substituted C₁-C₆ |
| | | | alkyl & an unsubstituted C₁-C₆ |
| | | | haloalkyl |
| 2 | 3 | halogen & halogen | halogen, halogen & halogen |
| 2 | 3 | halogen & halogen | halogen, halogen & an optionally |
| | | | substituted C₁-C₆ alkyl |
| 2 | 3 | halogen & halogen | halogen, halogen & an unsubstituted |
| | | | C₁-C₆ haloalkyl |
| 2 | 3 | halogen & halogen | halogen, an optionally substituted C₁-C₆ |
| | | | alkyl & an optionally substituted |
| | | | C₁-C₆ alkyl |
| 2 | 3 | halogen & halogen | halogen, an unsubstituted C₁-C₆ |
| | | | haloalkyl & an unsubstituted C₁-C₆ |
| | | | haloalkyl |
| 2 | 3 | halogen & halogen | an optionally substituted C₁-C₆ alkyl, |
| | | | an optionally substituted C₁-C₆ alkyl |
| | | | & an unsubstituted C₁-C₆ haloalkyl |
| 2 | 3 | halogen & halogen | an optionally substituted C₁-C₆ alkyl, |
| | | | an unsubstituted C₁-C₆ haloalkyl & an |
| | | | unsubstituted C₁-C₆ haloalkyl |
| 2 | 3 | halogen & an optionally substituted | halogen, an optionally substituted C₁-C₆ |
| | | C₁-C₆ alkyl | alkyl & an unsubstituted C₁-C₆ |
| | | | haloalkyl |
| 2 | 3 | halogen & an optionally substituted | halogen, halogen & halogen |
| | | C₁-C₆ alkyl | |
| 2 | 3 | halogen & an optionally substituted | halogen, halogen & an optionally |
| | | C₁-C₆ alkyl | substituted C₁-C₆ alkyl |
| 2 | 3 | halogen & an optionally substituted | halogen, halogen & an unsubstituted |
| | | C₁-C₆ alkyl | C₁-C₆ haloalkyl |
| 2 | 3 | halogen & an optionally substituted | halogen, an optionally substituted C₁-C₆ |
| | | C₁-C₆ alkyl | alkyl & an optionally substituted |
| | | | C₁-C₆ alkyl |
| 2 | 3 | halogen & an optionally substituted | halogen, an unsubstituted C₁-C₆ |
| | | C₁-C₆ alkyl | haloalkyl & an unsubstituted C₁-C₆ |
| | | | haloalkyl |
| 2 | 3 | halogen & an optionally substituted | an optionally substituted C₁-C₆ alkyl, |
| | | C₁-C₆ alkyl | an optionally substituted C₁-C₆ alkyl |
| | | | & an unsubstituted C₁-C₆ haloalkyl |
| 2 | 3 | halogen & an optionally substituted | an optionally substituted C₁-C₆ alkyl, |
| | | C₁-C₆ alkyl | an unsubstituted C₁-C₆ haloalkyl & an |
| | | | unsubstituted C₁-C₆ haloalkyl |
| 2 | 3 | halogen & an unsubstituted C₁-C₆ | halogen, an optionally substituted C₁-C₆ |
| | | haloalkyl | alkyl & an unsubstituted C₁-C₆ |
| | | | haloalkyl |
| 2 | 3 | halogen & an unsubstituted C₁-C₆ | halogen, halogen & halogen |
| | | haloalkyl | |
| 2 | 3 | halogen & an unsubstituted C₁-C₆ | halogen, halogen & an optionally |
| | | haloalkyl | substituted C₁-C₆ alkyl |
| 2 | 3 | halogen & an unsubstituted C₁-C₆ | halogen, halogen & an unsubstituted |
| | | haloalkyl | C₁-C₆ haloalkyl |
| 2 | 3 | halogen & an unsubstituted C₁-C₆ | halogen, an optionally substituted C₁-C₆ |
| | | haloalkyl | alkyl & an optionally substituted |
| | | | C₁-C₆ alkyl |
| 2 | 3 | halogen & an unsubstituted C₁-C₆ | halogen, an unsubstituted C₁-C₆ |
| | | haloalkyl | haloalkyl & an unsubstituted C₁-C₆ |
| | | | haloalkyl |
| 2 | 3 | halogen & an unsubstituted C₁-C₆ | an optionally substituted C₁-C₆ alkyl, |
| | | haloalkyl | an optionally substituted C₁-C₆ alkyl |
| | | | & an unsubstituted C₁-C₆ haloalkyl |
| 2 | 3 | halogen & an unsubstituted C₁-C₆ | an optionally substituted C₁-C₆ alkyl, |
| | | haloalkyl | an unsubstituted C₁-C₆ haloalkyl & an |
| | | | unsubstituted C₁-C₆ haloalkyl |
| 2 | 3 | an optionally substituted C₁-C₆ alkyl | halogen, an optionally substituted C₁-C₆ |
| | | & an optionally substituted C₁-C₆ | alkyl & an unsubstituted C₁-C₆ |
| | | alkyl | haloalkyl |
| 2 | 3 | an optionally substituted C₁-C₆ alkyl | halogen, halogen & halogen |
| | | & an optionally substituted C₁-C₆ | |
| | | alkyl | |
| 2 | 3 | an optionally substituted C₁-C₆ alkyl | halogen, halogen & an optionally |
| | | & an optionally substituted C₁-C₆ | substituted C₁-C₆ alkyl |
| | | alkyl | |
| 2 | 3 | an optionally substituted C₁-C₆ alkyl | halogen, halogen & an unsubstituted |
| | | & an optionally substituted C₁-C₆ | C₁-C₆ haloalkyl |
| | | alkyl | |
| 2 | 3 | an optionally substituted C₁-C₆ alkyl | halogen, an optionally substituted C₁-C₆ |
| | | & an optionally substituted C₁-C₆ | alkyl & an optionally substituted |
| | | alkyl | C₁-C₆ alkyl |
| 2 | 3 | an optionally substituted C₁-C₆ alkyl | halogen, an unsubstituted C₁-C₆ |
| | | & an optionally substituted C₁-C₆ | haloalkyl & an unsubstituted C₁-C₆ |
| | | alkyl | haloalkyl |
| 2 | 3 | an optionally substituted C₁-C₆ alkyl | an optionally substituted C₁-C₆ alkyl, |
| | | & an optionally substituted C₁-C₆ | an optionally substituted C₁-C₆ alkyl |
| | | alkyl | & an unsubstituted C₁-C₆ haloalkyl |
| 2 | 3 | an optionally substituted C₁-C₆ alkyl | an optionally substituted C₁-C₆ alkyl, |
| | | & an optionally substituted C₁-C₆ | an unsubstituted C₁-C₆ haloalkyl & an |
| | | alkyl | unsubstituted CC₁-C₆ haloalkyl |
| 2 | 3 | an optionally substituted C₁-C₆ alkyl | halogen, an optionally substituted C₁-C₆ |
| | | & an unsubstituted C₁-C₆ haloalkyl | alkyl & an unsubstituted C₁-C₆ |
| | | | haloalkyl |
| 2 | 3 | an optionally substituted C₁-C₆ alkyl | halogen, halogen & halogen |
| | | & an unsubstituted C₁-C₆ haloalkyl | |
| 2 | 3 | an optionally substituted C₁-C₆ alkyl | halogen, halogen & an optionally |
| | | & an unsubstituted C₁-C₆ haloalkyl | substituted C₁-C₆ alkyl |
| 2 | 3 | an optionally substituted C₁-C₆ alkyl | halogen, halogen & an unsubstituted |
| | | & an unsubstituted C₁-C₆ haloalkyl | C₁-C₆ haloalkyl |
| 2 | 3 | an optionally substituted C₁-C₆ alkyl | halogen, an optionally substituted C₁-C₆ |
| | | & an unsubstituted C₁-C₆ haloalkyl | alkyl & an optionally substituted |
| | | | C₁-C₆ alkyl |
| 2 | 3 | an optionally substituted C₁-C₆ alkyl | halogen, an unsubstituted C₁-C₆ |
| | | & an unsubstituted C₁-C₆ haloalkyl | haloalkyl & an unsubstituted C₁-C₆ |
| | | | haloalkyl |
| 2 | 3 | an optionally substituted C₁-C₆ alkyl | an optionally substituted C₁-C₆ alkyl, |
| | | & an unsubstituted C₁-C₆ haloalkyl | an optionally substituted C₁-C₆ alkyl |
| | | | & an unsubstituted C₁-C₆ haloalkyl |
| 2 | 3 | an optionally substituted C₁-C₆ alkyl | an optionally substituted C₁-C₆ alkyl, |
| | | & an unsubstituted C₁-C₆ haloalkyl | an unsubstituted C₁-C₆ haloalkyl & an |
| | | | unsubstituted C₁-C₆ haloalkyl |
| 2 | 3 | an unsubstituted C₁-C₆ haloalkyl & | halogen, halogen & halogen |
| | | an unsubstituted C₁-C₆ haloalkyl | |
| 2 | 3 | an unsubstituted C₁-C₆ haloalkyl & | halogen, halogen & an optionally |
| | | an unsubstituted C₁-C₆ haloalkyl | substituted C₁-C₆ alkyl |
| 2 | 3 | an unsubstituted C₁-C₆ haloalkyl & | halogen, halogen & an unsubstituted |
| | | an unsubstituted C₁-C₆ haloalkyl | C₁-C₆ haloalkyl |
| 2 | 3 | an unsubstituted C₁-C₆ haloalkyl & | halogen, an optionally substituted C₁-C₆ |
| | | an unsubstituted C₁-C₆ haloalkyl | alkyl & an optionally substituted |
| | | | C₁-C₆ alkyl |
| 2 | 3 | an unsubstituted C₁-C₆ haloalkyl & | halogen, an unsubstituted C₁-C₆ |
| | | an unsubstituted C₁-C₆ haloalkyl | haloalkyl & an unsubstituted C₁-C₆ |
| | | | haloalkyl |
| 2 | 3 | an unsubstituted C₁-C₆ haloalkyl & | an optionally substituted C₁-C₆ alkyl, |
| | | an unsubstituted C₁-C₆ haloalkyl | an optionally substituted C₁-C₆ alkyl |
| | | | & an unsubstituted C₁-C₆ haloalkyl |
| 2 | 3 | an unsubstituted C₁-C₆ haloalkyl & | an optionally substituted C₁-C₆ alkyl, |
| | | an unsubstituted C₁-C₆ haloalkyl | an unsubstituted C₁-C₆ haloalkyl & an |
| | | | unsubstituted C₁-C₆ haloalkyl |
| 2 | 3 | —CN and halogen | halogen, halogen & halogen |
| 2 | 3 | —CN and halogen | halogen, halogen & an optionally |
| | | | substituted C₁-C₆ alkyl |
| 2 | 3 | —CN and halogen | halogen, halogen & an unsubstituted |
| | | | C₁-C₆ haloalkyl |
| 2 | 3 | —CN and halogen | halogen, an optionally substituted C₁-C₆ |
| | | | alkyl & an optionally substituted |
| | | | C₁-C₆ alkyl |
| 2 | 3 | —CN and halogen | halogen, an unsubstituted C₁-C₆ |
| | | | haloalkyl & an unsubstituted C₁-C₆ |
| | | | haloalkyl |
| 2 | 3 | —CN and halogen | an optionally substituted C₁-C₆ alkyl, |
| | | | an optionally substituted C₁-C₆ alkyl |
| | | | & an unsubstituted C₁-C₆ haloalkyl |
| 2 | 3 | —CN and halogen | an optionally substituted C₁-C₆ alkyl, |
| | | | an unsubstituted C₁-C₆ haloalkyl & an |
| | | | unsubstituted C₁-C₆ haloalkyl |
| 2 | 3 | —CN & an optionally substituted C₁-C₆ alkyl | halogen, halogen & halogen |
| 2 | 3 | —CN & an optionally substituted C₁-C₆ alkyl | halogen, halogen & an optionally |
| | | | substituted C₁-C₆ alkyl |
| 2 | 3 | —CN & an optionally substituted C₁-C₆ alkyl | halogen, halogen & an unsubstituted |
| | | | C₁-C₆ haloalkyl |
| 2 | 3 | —CN & an optionally substituted C₁-C₆ alkyl | halogen, an optionally substituted C₁-C₆ |
| | | | alkyl & an optionally substituted |
| | | | C₁-C₆ alkyl |
| 2 | 3 | —CN & an optionally substituted C₁-C₆ alkyl | halogen, an unsubstituted C₁-C₆ |
| | | | haloalkyl & an unsubstituted C₁-C₆ |
| | | | haloalkyl |
| 2 | 3 | —CN & an optionally substituted C₁-C₆ alkyl | an optionally substituted C₁-C₆ alkyl, |
| | | | an optionally substituted C₁-C₆ alkyl |
| | | | & an unsubstituted C₁-C₆ haloalkyl |
| 2 | 3 | —CN & an optionally substituted C₁-C₆ alkyl | an optionally substituted C₁-C₆ alkyl, |
| | | | an unsubstituted C₁-C₆ haloalkyl & an |
| | | | unsubstituted C₁-C₆ haloalkyl |
| 2 | 3 | —CN & an unsubstituted C₁-C₆ | halogen, halogen & halogen |
| | | haloalkyl | |
| 2 | 3 | —CN & an unsubstituted C₁-C₆ | halogen, halogen & an optionally |
| | | haloalkyl | substituted C₁-C₆alkyl |
| 2 | 3 | —CN & an unsubstituted C₁-C₆ | halogen, halogen & an unsubstituted |
| | | haloalkyl | C₁-C₆ haloalkyl |
| 2 | 3 | —CN & an unsubstituted C₁-C₆ | halogen, an optionally substituted C₁-C₆ |
| | | haloalkyl | alkyl & an optionally substituted |
| | | | C₁-C₆ alkyl |
| 2 | 3 | —CN & an unsubstituted C₁-C₆ | halogen, an unsubstituted C₁-C₆ |
| | | haloalkyl | haloalkyl & an unsubstituted C₁-C₆ |
| | | | haloalkyl |
| 2 | 3 | —CN & an unsubstituted C₁-C₆ | an optionally substituted C₁-C₆ alkyl, |
| | | haloalkyl | an optionally substituted C₁-C₆ alkyl |
| | | | & an unsubstituted C₁-C₆ haloalkyl |
| 2 | 3 | —CN & an unsubstituted C₁-C₆ | an optionally substituted C₁-C₆ alkyl, |
| | | haloalkyl | an unsubstituted C₁-C₆ haloalkyl & an |
| | | | unsubstituted C₁-C₆ haloalkyl |
| 3 | 3 | halogen, halogen & halogen | halogen, halogen & halogen |
| 3 | 3 | halogen, halogen & halogen | halogen, halogen & an optionally |
| | | | substituted C₁-C₆ alkyl |
| 3 | 3 | halogen, halogen & halogen | halogen, halogen & an unsubstituted |
| | | | C₁-C₆ haloalkyl |
| 3 | 3 | halogen, halogen & halogen | halogen, an optionally substituted C₁-C₆ |
| | | | alkyl & an optionally substituted |
| | | | C₁-C₆ alkyl |
| 3 | 3 | halogen, halogen & halogen | halogen, an unsubstituted C₁-C₆ |
| | | | haloalkyl & an unsubstituted C₁-C₆ |
| | | | haloalkyl |
| 3 | 3 | halogen, halogen & halogen | an optionally substituted C₁-C₆ alkyl, |
| | | | an optionally substituted C₁-C₆ alkyl |
| | | | & an unsubstituted C₁-C₆ haloalkyl |
| 3 | 3 | halogen, halogen & halogen | an optionally substituted C₁-C₆ alkyl, |
| | | | an unsubstituted C₁-C₆ haloalkyl & an |
| | | | unsubstituted C₁-C₆ haloalkyl |
| 3 | 3 | halogen, halogen & halogen | halogen, an optionally substituted C₁-C₆ |
| | | | alkyl & an unsubstituted C₁-C₆ |
| | | | haloalkyl |
| 3 | 3 | halogen, halogen & an optionally | halogen, halogen & halogen |
| | | substituted C₁-C₆ alkyl | |
| 3 | 3 | halogen, halogen & an optionally | halogen, halogen & an optionally |
| | | substituted C₁-C₆ alkyl | substituted C₁-C₆ alkyl |
| 3 | 3 | halogen, halogen & an optionally | halogen, halogen & an unsubstituted |
| | | substituted C₁-C₆ alkyl | C₁-C₆ haloalkyl |
| 3 | 3 | halogen, halogen & an optionally | halogen, an optionally substituted C₁-C₆ |
| | | substituted C₁-C₆ alkyl | alkyl & an optionally substituted |
| | | | C₁-C₆ alkyl |
| 3 | 3 | halogen, halogen & an optionally | halogen, an unsubstituted C₁-C₆ |
| | | substituted C₁-C₆ alkyl | haloalkyl & an unsubstituted C₁-C₆ |
| | | | haloalkyl |
| 3 | 3 | halogen, halogen & an optionally | an optionally substituted C₁-C₆ alkyl, |
| | | substituted C₁-C₆ alkyl | an optionally substituted C₁-C₆ alkyl |
| | | | & an unsubstituted C₁-C₆ haloalkyl |
| 3 | 3 | halogen, halogen & an optionally | an optionally substituted C₁-C₆ alkyl, |
| | | substituted C₁-C₆ alkyl | an unsubstituted C₁-C₆ haloalkyl & an |
| | | | unsubstituted C₁-C₆ haloalkyl |
| 3 | 3 | halogen, halogen & an optionally | halogen, an optionally substituted C₁-C₆ |
| | | substituted C₁-C₆ alkyl | alkyl & an unsubstituted C₁-C₆ |
| | | | haloalkyl |
| 3 | 3 | halogen, halogen & an unsubstituted | halogen, halogen & halogen |
| | | C₁-C₆ haloalkyl | |
| 3 | 3 | halogen, halogen & an unsubstituted | halogen, halogen & an optionally |
| | | C₁-C₆ haloalkyl | substituted C₁-C₆ alkyl |
| 3 | 3 | halogen, halogen & an unsubstituted | halogen, halogen & an unsubstituted |
| | | C₁-C₆ haloalkyl | C₁-C₆ haloalkyl |
| 3 | 3 | halogen, halogen & an unsubstituted | halogen, an optionally substituted C₁-C₆ |
| | | C₁-C₆ haloalkyl | alkyl & an optionally substituted |
| | | | C₁-C₆ alkyl |
| 3 | 3 | halogen, halogen & an unsubstituted | halogen, an unsubstituted C₁-C₆ |
| | | C₁-C₆ haloalkyl | haloalkyl & an unsubstituted C₁-C₆ |
| | | | haloalkyl |
| 3 | 3 | halogen, halogen & an unsubstituted | an optionally substituted C₁-C₆ alkyl, |
| | | C₁-C₆ haloalkyl | an optionally substituted C₁-C₆ alkyl |
| | | | & an unsubstituted C₁-C₆ haloalkyl |
| 3 | 3 | halogen, halogen & an unsubstituted | an optionally substituted C₁-C₆ alkyl, |
| | | C₁-C₆ haloalkyl | an unsubstituted C₁-C₆ haloalkyl & an |
| | | | unsubstituted C₁-C₆ haloalkyl |
| 3 | 3 | halogen, halogen & an unsubstituted | halogen, an optionally substituted C₁-C₆ |
| | | C₁-C₆ haloalkyl | alkyl & an unsubstituted C₁-C₆ |
| | | | haloalkyl |
| 3 | 3 | halogen, an optionally substituted | halogen, halogen & halogen |
| | | C₁-C₆ alkyl & an optionally | |
| | | substituted C₁-C₆ alkyl | |
| 3 | 3 | halogen, an optionally substituted | halogen, halogen & an optionally |
| | | C₁-C₆ alkyl & an optionally | substituted C₁-C₆ alkyl |
| | | substituted C₁-C₆ alkyl | |
| 3 | 3 | halogen, an optionally substituted | halogen, halogen & an unsubstituted |
| | | C₁-C₆ alkyl & an optionally | C₁-C₆ haloalkyl |
| | | substituted C₁-C₆ alkyl | |
| 3 | 3 | halogen, an optionally substituted | halogen, an optionally substituted C₁-C₆ |
| | | C₁-C₆ alkyl & an optionally | alkyl & an optionally substituted |
| | | substituted C₁-C₆ alkyl | C₁-C₆ alkyl |
| 3 | 3 | halogen, an optionally substituted | halogen, an unsubstituted C₁-C₆ |
| | | C₁-C₆ alkyl & an optionally | haloalkyl & an unsubstituted C₁-C₆ |
| | | substituted C₁-C₆ alkyl | haloalkyl |
| 3 | 3 | halogen, an optionally substituted | an optionally substituted C₁-C₆ alkyl, |
| | | C₁-C₆ alkyl & an optionally | an optionally substituted C₁-C₆ alkyl |
| | | substituted C₁-C₆ alkyl | & an unsubstituted C₁-C₆ haloalkyl |
| 3 | 3 | halogen, an optionally substituted | an optionally substituted C₁-C₆ alkyl, |
| | | C₁-C₆ alkyl & an optionally | an unsubstituted C₁-C₆ haloalkyl & an |
| | | substituted C₁-C₆ alkyl | unsubstituted C₁-C₆ haloalkyl |
| 3 | 3 | halogen, an optionally substituted | halogen, an optionally substituted C₁-C₆ |
| | | C₁-C₆ alkyl & an optionally | alkyl & an unsubstituted C₁-C₆ |
| | | substituted C₁-C₆ alkyl | haloalkyl |
| 3 | 3 | halogen, an unsubstituted C₁-C₆ | halogen, halogen & halogen |
| | | haloalkyl & an unsubstituted C₁-C₆ | |
| | | haloalkyl | |
| 3 | 3 | halogen, an unsubstituted C₁-C₆ | halogen, halogen & an optionally |
| | | haloalkyl & an unsubstituted C₁-C₆ | substituted C₁-C₆ alkyl |
| | | haloalkyl | |
| 3 | 3 | halogen, an unsubstituted C₁-C₆ | halogen, halogen & an unsubstituted |
| | | haloalkyl & an unsubstituted C₁-C₆ | C₁-C₆ haloalkyl |
| | | haloalkyl | |
| 3 | 3 | halogen, an unsubstituted C₁-C₆ | halogen, an optionally substituted C₁-C₆ |
| | | haloalkyl & an unsubstituted C₁-C₆ | alkyl & an optionally substituted |
| | | haloalkyl | C₁-C₆ alkyl |
| 3 | 3 | halogen, an unsubstituted C₁-C₆ | halogen, an unsubstituted C₁-C₆ |
| | | haloalkyl & an unsubstituted C₁-C₆ | haloalkyl & an unsubstituted C₁-C₆ |
| | | haloalkyl | haloalkyl |
| 3 | 3 | halogen, an unsubstituted C₁-C₆ | an optionally substituted C₁-C₆ alkyl, |
| | | haloalkyl & an unsubstituted C₁-C₆ | an optionally substituted C₁-C₆ alkyl |
| | | haloalkyl | & an unsubstituted C₁-C₆ haloalkyl |
| 3 | 3 | halogen, an unsubstituted C₁-C₆ | an optionally substituted C₁-C₆ alkyl, |
| | | haloalkyl & an unsubstituted C₁-C₆ | an unsubstituted C₁-C₆ haloalkyl & an |
| | | haloalkyl | unsubstituted C₁-C₆ haloalkyl |
| 3 | 3 | an optionally substituted C₁-C₆ | halogen, halogen & halogen |
| | | alkyl, an optionally substituted C₁-C₆ alkyl | |
| | | & an unsubstituted C₁-C₆ | |
| | | haloalkyl | |
| 3 | 3 | an optionally substituted C₁-C₆ | halogen, halogen & an optionally |
| | | alkyl, an optionally substituted C₁-C₆ alkyl | substituted C₁-C₆ alkyl |
| | | & an unsubstituted C₁-C₆ | |
| | | haloalkyl | |
| 3 | 3 | an optionally substituted C₁-C₆ | halogen, halogen & an unsubstituted |
| | | alkyl, an optionally substituted C₁-C₆ alkyl | C₁-C₆ haloalkyl |
| | | & an unsubstituted C₁-C₆ | |
| | | haloalkyl | |
| 3 | 3 | an optionally substituted C₁-C₆ | halogen, an optionally substituted C₁-C₆ |
| | | alkyl, an optionally substituted C₁-C₆ alkyl | alkyl & an optionally substituted |
| | | & an unsubstituted C₁-C₆ | C₁-C₆ alkyl |
| | | haloalkyl | |
| 3 | 3 | an optionally substituted C₁-C₆ | halogen, an unsubstituted C₁-C₆ |
| | | alkyl, an optionally substituted C₁-C₆ alkyl | haloalkyl & an unsubstituted C₁-C₆ |
| | | & an unsubstituted C₁-C₆ | haloalkyl |
| | | haloalkyl | |
| 3 | 3 | an optionally substituted C₁-C₆ | an optionally substituted C₁-C₆ alkyl, |
| | | alkyl, an optionally substituted C₁-C₆ alkyl | an optionally substituted C₁-C₆ alkyl |
| | | & an unsubstituted C₁-C₆ | & an unsubstituted C₁-C₆ haloalkyl |
| | | haloalkyl | |
| 3 | 3 | an optionally substituted C₁-C₆ | an optionally substituted C₁-C₆ alkyl, |
| | | alkyl, an optionally substituted C₁-C₆ alkyl | an unsubstituted C₁-C₆ haloalkyl & an |
| | | & an unsubstituted C₁-C₆ | unsubstituted C₁-C₆ haloalkyl |
| | | haloalkyl | |
| 3 | 3 | an optionally substituted C₁-C₆ | halogen, halogen & halogen |
| | | alkyl, an unsubstituted C₁-C₆ | |
| | | haloalkyl & an unsubstituted C₁-C₆ | |
| | | haloalkyl | |
| 3 | 3 | an optionally substituted C₁-C₆ | halogen, halogen & an optionally |
| | | alkyl, an unsubstituted C₁-C₆ | substituted C₁-C₆ alkyl |
| | | haloalkyl & an unsubstituted C₁-C₆ | |
| | | haloalkyl | |
| 3 | 3 | an optionally substituted C₁-C₆ | halogen, halogen & an unsubstituted |
| | | alkyl, an unsubstituted C₁-C₆ | C₁-C₆ haloalkyl |
| | | haloalkyl & an unsubstituted C₁-C₆ | |
| | | haloalkyl | |
| 3 | 3 | an optionally substituted C₁-C₆ | halogen, an optionally substituted C₁-C₆ |
| | | alkyl, an unsubstituted C₁-C₆ | alkyl & an optionally substituted |
| | | haloalkyl & an unsubstituted C₁-C₆ | C₁-C₆ alkyl |
| | | haloalkyl | |
| 3 | 3 | an optionally substituted C₁-C₆ | halogen, an unsubstituted C₁-C₆ |
| | | alkyl, an unsubstituted C₁-C₆ | haloalkyl & an unsubstituted C₁-C₆ |
| | | haloalkyl & an unsubstituted C₁-C₆ | haloalkyl |
| | | haloalkyl | |
| 3 | 3 | an optionally substituted C₁-C₆ | an optionally substituted C₁-C₆ alkyl, |
| | | alkyl, an unsubstituted C₁-C₆ | an optionally substituted C₁-C₆ alkyl |
| | | haloalkyl & an unsubstituted C₁-C₆ | & an unsubstituted C₁-C₆ haloalkyl |
| | | haloalkyl | |
| 3 | 3 | an optionally substituted C₁-C₆ | an optionally substituted C₁-C₆ alkyl, |
| | | alkyl, an unsubstituted C₁-C₆ | an unsubstituted C₁-C₆ haloalkyl & an |
| | | haloalkyl & an unsubstituted C₁-C₆ | unsubstituted C₁-C₆ haloalkyl |
| | | haloalkyl | |
| 3 | 3 | halogen, an optionally substituted | halogen, halogen & halogen |
| | | C₁-C₆ alkyl & an unsubstituted | |
| | | C₁-C₆ haloalkyl | |
| 3 | 3 | halogen, an optionally substituted | halogen, halogen & an optionally |
| | | C₁-C₆ alkyl & an unsubstituted | substituted C₁-C₆ alkyl |
| | | C₁-C₆ haloalkyl | |
| 3 | 3 | halogen, an optionally substituted | halogen, halogen & an unsubstituted |
| | | C₁-C₆ alkyl & an unsubstituted | C₁-C₆ haloalkyl |
| | | C₁-C₆ haloalkyl | |
| 3 | 3 | halogen, an optionally substituted | halogen, an optionally substituted C₁-C₆ |
| | | C₁-C₆ alkyl & an unsubstituted | alkyl & an optionally substituted |
| | | C₁-C₆ haloalkyl | C₁-C₆ alkyl |
| 3 | 3 | halogen, an optionally substituted | halogen, an unsubstituted C₁-C₆ |
| | | C₁-C₆ alkyl & an unsubstituted | haloalkyl & an unsubstituted C₁-C₆ |
| | | C₁-C₆ haloalkyl | haloalkyl |
| 3 | 3 | halogen, an optionally substituted | an optionally substituted C₁-C₆ alkyl, |
| | | C₁-C₆ alkyl & an unsubstituted | an optionally substituted C₁-C₆ alkyl |
| | | C₁-C₆ haloalkyl | & an unsubstituted C₁-C₆ haloalkyl |
| 3 | 3 | halogen, an optionally substituted | an optionally substituted C₁-C₆ alkyl, |
| | | C₁-C₆ alkyl & an unsubstituted | an unsubstituted C₁-C₆ haloalkyl & an |
| | | C₁-C₆ haloalkyl | unsubstituted C₁-C₆ haloalkyl |
| 3 | 3 | —CN, halogen and halogen | halogen, halogen & halogen |
| 3 | 3 | —CN, halogen and halogen | halogen, halogen & an optionally |
| | | | substituted C₁-C₆ alkyl |
| 3 | 3 | —CN, halogen and halogen | halogen, halogen & an unsubstituted |
| | | | C₁-C₆ haloalkyl |
| 3 | 3 | —CN, halogen and halogen | halogen, an optionally substituted C₁-C₆ |
| | | | alkyl & an optionally substituted |
| | | | C₁-C₆ alkyl |
| 3 | 3 | —CN, halogen and halogen | halogen, an unsubstituted C₁-C₆ |
| | | | haloalkyl & an unsubstituted C₁-C₆ |
| | | | haloalkyl |
| 3 | 3 | —CN, halogen and halogen | an optionally substituted C₁-C₆ alkyl, |
| | | | an optionally substituted C₁-C₆ alkyl |
| | | | & an unsubstituted C₁-C₆ haloalkyl |
| 3 | 3 | —CN, halogen and halogen | an optionally substituted C₁-C₆ alkyl, |
| | | | an unsubstituted C₁-C₆ haloalkyl & an |
| | | | unsubstituted C₁-C₆ haloalkyl |
| 3 | 3 | —CN, an optionally substituted C₁-C₆ | halogen, halogen & halogen |
| | | alkyl & an optionally substituted C₁-C₆ alkyl | |
| 3 | 3 | —CN, an optionally substituted C₁-C₆ | halogen, halogen & an optionally |
| | | alkyl & an optionally substituted C₁-C₆ alkyl | substituted C₁-C₆ alkyl |
| 3 | 3 | —CN, an optionally substituted C₁-C₆ | halogen, halogen & an unsubstituted |
| | | alkyl & an optionally substituted C₁-C₆ alkyl | C₁-C₆ haloalkyl |
| 3 | 3 | —CN, an optionally substituted C₁-C₆ | halogen, an optionally substituted C₁-C₆ |
| | | alkyl & an optionally substituted C₁-C₆ alkyl | alkyl & an optionally substituted |
| | | | C₁-C₆ alkyl |
| 3 | 3 | —CN, an optionally substituted C₁-C₆ | halogen, an unsubstituted C₁-C₆ |
| | | alkyl & an optionally substituted C₁-C₆ alkyl | haloalkyl & an unsubstituted C₁-C₆ |
| | | | haloalkyl |
| 3 | 3 | —CN, an optionally substituted C₁-C₆ | an optionally substituted C₁-C₆ alkyl, |
| | | alkyl & an optionally substituted C₁-C₆ alkyl | an optionally substituted C₁-C₆ alkyl |
| | | | & an unsubstituted C₁-C₆ haloalkyl |
| 3 | 3 | —CN, an optionally substituted C₁-C₆ | an optionally substituted C₁-C₆ alkyl, |
| | | alkyl & an optionally substituted C₁-C₆ alkyl | an unsubstituted C₁-C₆ haloalkyl & an |
| | | | unsubstituted C₁-C₆ haloalkyl |
| 3 | 3 | —CN, an optionally substituted C₁-C₆ | halogen, halogen & halogen |
| | | alkyl & an unsubstituted C₁-C₆ | |
| | | haloalkyl | |
| 3 | 3 | -CN, an optionally substituted C₁-C₆ | halogen, halogen & an optionally |
| | | alkyl & an unsubstituted C₁-C₆ | substituted C₁-C₆ alkyl |
| | | haloalkyl | |
| 3 | 3 | —CN, an optionally substituted C₁-C₆ | halogen, halogen & an unsubstituted |
| | | alkyl & an unsubstituted C₁-C₆ | C₁-C₆ haloalkyl |
| | | haloalkyl | |
| 3 | 3 | —CN, an optionally substituted C₁-C₆ | halogen, an optionally substituted C₁-C₆ |
| | | alkyl & an unsubstituted C₁-C₆ | alkyl & an optionally substituted |
| | | haloalkyl | C₁-C₆ alkyl |
| 3 | 3 | —CN, an optionally substituted C₁-C₆ | halogen, an unsubstituted C₁-C₆ |
| | | alkyl & an unsubstituted C₁-C₆ | haloalkyl & an unsubstituted C₁-C₆ |
| | | haloalkyl | haloalkyl |
| 3 | 3 | —CN, an optionally substituted C₁-C₆ | an optionally substituted C₁-C₆ alkyl, |
| | | alkyl & an unsubstituted C₁-C₆ | an optionally substituted C₁-C₆ alkyl |
| | | haloalkyl | & an unsubstituted C₁-C₆ haloalkyl |
| 3 | 3 | —CN, an optionally substituted C₁-C₆ | an optionally substituted C₁-C₆ alkyl, |
| | | alkyl & an unsubstituted C₁-C₆ | an unsubstituted C₁-C₆ haloalkyl & an |
| | | haloalkyl | unsubstituted C₁-C₆ haloalkyl |
| 3 | 3 | —CN, an unsubstituted C₁-C₆ | halogen, halogen & halogen |
| | | haloalkyl & an unsubstituted C₁-C₆ | |
| | | haloalkyl | |
| 3 | 3 | —CN, an unsubstituted C₁-C₆ | halogen, halogen & an optionally |
| | | haloalkyl & an unsubstituted C₁-C₆ | substituted C₁-C₆ alkyl |
| | | haloalkyl | |
| 3 | 3 | —CN, an unsubstituted C₁-C₆ | halogen, halogen & an unsubstituted |
| | | haloalkyl & an unsubstituted C₁-C₆ | C₁-C₆ haloalkyl |
| | | haloalkyl | |
| 3 | 3 | —CN, an unsubstituted C₁-C₆ | halogen, an optionally substituted C₁-C₆ |
| | | haloalkyl & an unsubstituted C₁-C₆ | alkyl & an optionally substituted |
| | | haloalkyl | C₁-C₆ alkyl |
| 3 | 3 | —CN, an unsubstituted C₁-C₆ | halogen, an unsubstituted C₁-C₆ |
| | | haloalkyl & an unsubstituted C₁-C₆ | haloalkyl & an unsubstituted C₁-C₆ |
| | | haloalkyl | haloalkyl |
| 3 | 3 | —CN, an unsubstituted C₁-C₆ | an optionally substituted C₁-C₆ alkyl, |
| | | haloalkyl & an unsubstituted C₁-C₆ | an optionally substituted C₁-C₆ alkyl |
| | | haloalkyl | & an unsubstituted C₁-C₆ haloalkyl |
| 3 | 3 | —CN, an unsubstituted C₁-C₆ | an optionally substituted C₁-C₆ alkyl, |
| | | haloalkyl & an unsubstituted C₁-C₆ | an unsubstituted C₁-C₆ haloalkyl & an |
| | | haloalkyl | unsubstituted C₁-C₆ haloalkyl |
| 3 | 3 | —CN, halogen, & an optionally | halogen, halogen & halogen |
| | | substituted C₁-C₆ alkyl | |
| 3 | 3 | —CN, halogen, & an optionally | halogen, halogen & an optionally |
| | | substituted C₁-C₆ alkyl | substituted C₁-C₆ alkyl |
| 3 | 3 | —CN, halogen, & an optionally | halogen, halogen & an unsubstituted |
| | | substituted C₁-C₆ alkyl | C₁-C₆ haloalkyl |
| 3 | 3 | —CN, halogen, & an optionally | halogen, an optionally substituted C₁-C₆ |
| | | substituted C₁-C₆ alkyl | alkyl & an optionally substituted |
| | | | C₁-C₆ alkyl |
| 3 | 3 | —CN, halogen, & an optionally | halogen, an unsubstituted C₁-C₆ |
| | | substituted C₁-C₆ alkyl | haloalkyl & an unsubstituted C₁-C₆ |
| | | | haloalkyl |
| 3 | 3 | —CN, halogen, & an optionally | an optionally substituted C₁-C₆ alkyl, |
| | | substituted C₁-C₆ alkyl | an optionally substituted C₁-C₆ alkyl |
| | | | & an unsubstituted C₁-C₆ haloalkyl |
| 3 | 3 | —CN, halogen, & an optionally | an optionally substituted C₁-C₆ alkyl, |
| | | substituted C₁-C₆ alkyl | an unsubstituted C₁-C₆ haloalkyl & an |
| | | | unsubstituted C₁-C₆ haloalkyl |
| 3 | 3 | —CN, halogen, & an unsubstituted | halogen, halogen & halogen |
| | | C₁-C₆ haloalkyl | |
| 3 | 3 | —CN, halogen, & an unsubstituted | halogen, halogen & an optionally |
| | | C₁-C₆ haloalkyl | substituted C₁-C₆ alkyl |
| 3 | 3 | —CN, halogen, & an unsubstituted | halogen, halogen & an unsubstituted |
| | | C₁-C₆ haloalkyl | C₁-C₆ haloalkyl |
| 3 | 3 | —CN, halogen, & an unsubstituted | halogen, an optionally substituted C₁-C₆ |
| | | C₁-C₆ haloalkyl | alkyl & an optionally substituted |
| | | | C₁-C₆ alkyl |
| 3 | 3 | —CN, halogen, & an unsubstituted | halogen, an unsubstituted C₁-C₆ |
| | | C₁-C₆ haloalkyl | haloalkyl & an unsubstituted C₁-C₆ |
| | | | haloalkyl |
| 3 | 3 | —CN, halogen, & an unsubstituted | an optionally substituted C₁-C₆ alkyl, |
| | | C₁-C₆ haloalkyl | an optionally substituted C₁-C₆ alkyl |
| | | | & an unsubstituted C₁-C₆ haloalkyl |
| 3 | 3 | —CN, halogen, & an unsubstituted | an optionally substituted C₁-C₆ alkyl, |
| | | C₁-C₆ haloalkyl | an unsubstituted C₁-C₆ haloalkyl & an |
| | | | unsubstituted C₁-C₆ haloalkyl |
| 2 | 1 | halogen & halogen | halogen |
| 2 | 1 | halogen & halogen | an optionally substituted C₁-C₆ alkyl |
| 2 | 1 | halogen & halogen | an unsubstituted C₁-C₆ haloalkyl |
| 2 | 1 | halogen & an optionally substituted | halogen |
| | | C₁-C₆ alkyl | |
| 2 | 1 | halogen & an optionally substituted | an optionally substituted C₁-C₆ alkyl |
| | | C₁-C₆ alkyl | |
| 2 | 1 | halogen & an optionally substituted | an unsubstituted C₁-C₆ haloalkyl |
| | | C₁-C₆ alkyl | |
| 2 | 1 | halogen & an unsubstituted C₁-C₆ | halogen |
| | | haloalkyl | |
| 2 | 1 | halogen & an unsubstituted C₁-C₆ | an optionally substituted C₁-C₆ alkyl |
| | | haloalkyl | |
| 2 | 1 | halogen & an unsubstituted C₁-C₆ | an unsubstituted C₁-C₆ haloalkyl |
| | | haloalkyl | |
| 2 | 1 | halogen & —CN | halogen |
| 2 | 1 | halogen & —CN | an optionally substituted C₁-C₆ alkyl |
| 2 | 1 | halogen & —CN | an unsubstituted C₁-C₆ haloalkyl |
| 2 | 1 | —CN & an optionally substituted C₁-C₆ alkyl | halogen |
| 2 | 1 | —CN & an optionally substituted C₁-C₆ alkyl | an optionally substituted C₁-C₆ alkyl |
| 2 | 1 | —CN & an optionally substituted C₁-C₆ | an unsubstituted C₁-C₆ haloalkyl |
| | | alkyl | |
| 2 | 1 | —CN & unsubstituted C₁-C₆ haloalkyl | halogen |
| 2 | 1 | —CN & unsubstituted C₁-C₆ haloalkyl | an optionally substituted C₁-C₆ alkyl |
| 2 | 1 | —CN & unsubstituted C₁-C₆ haloalkyl | an unsubstituted C₁-C₆ haloalkyl |
| 2 | 1 | an optionally substituted C₁-C₆ alkyl | halogen |
| | | & an optionally substituted C₁-C₆ | |
| | | alkyl | |
| 2 | 1 | an optionally substituted C₁-C₆ alkyl | an optionally substituted C₁-C₆ alkyl |
| | | & an optionally substituted C₁-C₆ | |
| | | alkyl | |
| 2 | 1 | an optionally substituted C₁-C₆ alkyl | an unsubstituted C₁-C₆ haloalkyl |
| | | & an optionally substituted C₁-C₆ | |
| | | alkyl | |
| 2 | 1 | an optionally substituted C₁-C₆ alkyl | halogen |
| | | & unsubstituted C₁-C₆ haloalkyl | |
| 2 | 1 | an optionally substituted C₁-C₆ alkyl | an optionally substituted C₁-C₆ alkyl |
| | | & unsubstituted C₁-C₆ haloalkyl | |
| 2 | 1 | an optionally substituted C₁-C₆ alkyl | an unsubstituted C₁-C₆ haloalkyl |
| | | & unsubstituted C₁-C₆ haloalkyl | |
| 2 | 1 | un substituted C₁-C₆ haloalkyl & | halogen |
| | | unsubstituted C₁-C₆ haloalkyl | |
| 2 | 1 | unsubstituted C₁-C₆ haloalkyl & | an optionally substituted C₁-C₆ alkyl |
| | | unsubstituted C₁-C₆ haloalkyl | |
| 2 | 1 | unsubstituted C₁-C₆ haloalkyl & | an unsubstituted C₁-C₆ haloalkyl |
| | | unsubstituted C₁-C₆ haloalkyl | |
| 3 | 1 | halogen, halogen & halogen | halogen |
| 3 | 1 | halogen, halogen & halogen | an optionally substituted C₁-C₆ alkyl |
| 3 | 1 | halogen, halogen & halogen | an unsubstituted C₁-C₆ haloalkyl |
| 3 | 1 | halogen, halogen & an optionally | halogen |
| | | substituted C₁-C₆ alkyl | |
| 3 | 1 | halogen, halogen & an optionally | an optionally substituted C₁-C₆ alkyl |
| | | substituted C₁-C₆ alkyl | |
| 3 | 1 | halogen, halogen & an optionally | an unsubstituted C₁-C₆ haloalkyl |
| | | substituted C₁-C₆ alkyl | |
| 3 | 1 | halogen, halogen & an unsubstituted | halogen |
| | | C₁-C₆ haloalkyl | |
| 3 | 1 | halogen, halogen & an unsubstituted | an optionally substituted C₁-C₆ alkyl |
| | | C₁-C₆ haloalkyl | |
| 3 | 1 | halogen, halogen & an unsubstituted | an unsubstituted C₁-C₆ haloalkyl |
| | | C₁-C₆ haloalkyl | |
| 3 | 1 | halogen, an optionally substituted | halogen |
| | | C₁-C₆ alkyl & an optionally | |
| | | substituted C₁-C₆ alkyl | |
| 3 | 1 | halogen, an optionally substituted | an optionally substituted C₁-C₆ alkyl |
| | | C₁-C₆ alkyl & an optionally | |
| | | substituted C₁-C₆ alkyl | |
| 3 | 1 | halogen, an optionally substituted | an unsubstituted C₁-C₆ haloalkyl |
| | | C₁-C₆ alkyl & an optionally | |
| | | substituted C₁-C₆ alkyl | |
| 3 | 1 | halogen, an unsubstituted C₁-C₆ | halogen |
| | | haloalkyl & an unsubstituted C₁-C₆ | |
| | | haloalkyl | |
| 3 | 1 | halogen, an unsubstituted C₁-C₆ | an optionally substituted C₁-C₆ alkyl |
| | | haloalkyl & an unsubstituted C₁-C₆ | |
| | | haloalkyl | |
| 3 | 1 | halogen, an unsubstituted C₁-C₆ | an unsubstituted C₁-C₆ haloalkyl |
| | | haloalkyl & an unsubstituted C₁-C₆ | |
| | | haloalkyl | |
| 3 | 1 | an optionally substituted C₁-C₆ | halogen |
| | | alkyl, an optionally substituted C₁-C₆ alkyl | |
| | | & an unsubstituted C₁-C₆ | |
| | | haloalkyl | |
| 3 | 1 | an optionally substituted C₁-C₆ | an optionally substituted C₁-C₆ alkyl |
| | | alkyl, an optionally substituted C₁-C₆ alkyl | |
| | | & an unsubstituted C₁-C₆ | |
| | | haloalkyl | |
| 3 | 1 | an optionally substituted C₁-C₆ | an unsubstituted C₁-C₆ haloalkyl |
| | | alkyl, an optionally substituted C₁-C₆ alkyl | |
| | | & an unsubstituted C₁-C₆ | |
| | | haloalkyl | |
| 3 | 1 | an optionally substituted C₁-C₆ | halogen |
| | | alkyl, an unsubstituted C₁-C₆ | |
| | | haloalkyl & an unsubstituted C₁-C₆ | |
| | | haloalkyl | |
| 3 | 1 | an optionally substituted C₁-C₆ | an optionally substituted C₁-C₆ alkyl |
| | | alkyl, an unsubstituted C₁-C₆ | |
| | | haloalkyl & an unsubstituted C₁-C₆ | |
| | | haloalkyl | |
| 3 | 1 | an optionally substituted C₁-C₆ | an unsubstituted C₁-C₆ haloalkyl |
| | | alkyl, an unsubstituted C₁-C₆ | |
| | | haloalkyl & an unsubstituted C₁-C₆ | |
| | | haloalkyl | |
| 3 | 1 | halogen, an optionally substituted | halogen |
| | | C₁-C₆ alkyl & an unsubstituted | |
| | | C₁-C₆ haloalkyl | |
| 3 | 1 | halogen, an optionally substituted | an optionally substituted C₁-C₆ alkyl |
| | | C₁-C₆ alkyl & an unsubstituted | |
| | | C₁-C₆ haloalkyl | |
| 3 | 1 | halogen, an optionally substituted | an unsubstituted C₁-C₆ haloalkyl |
| | | C₁-C₆ alkyl & an unsubstituted | |
| | | C₁-C₆ haloalkyl | |
| 3 | 1 | —CN, halogen and halogen | halogen |
| 3 | 1 | —CN, halogen and halogen | an optionally substituted C₁-C₆ alkyl |
| 3 | 1 | —CN, halogen and halogen | an unsubstituted C₁-C₆ haloalkyl |
| 3 | 1 | —CN, an optionally substituted C₁-C₆ | halogen |
| | | alkyl & an optionally substituted C₁-C₆ alkyl | |
| 3 | 1 | —CN, an optionally substituted C₁-C₆ | an optionally substituted C₁-C₆ alkyl |
| | | alkyl & an optionally substituted C₁-C₆ alkyl | |
| 3 | 1 | —CN, an optionally substituted C₁-C₆ | an unsubstituted C₁-C₆ haloalkyl |
| | | alkyl & an optionally substituted C₁-C₆ alkyl | |
| 3 | 1 | —CN, an optionally substituted C₁-C₆ | halogen |
| | | alkyl & an unsubstituted C₁-C₆ | |
| | | haloalkyl | |
| 3 | 1 | —CN, an optionally substituted C₁-C₆ | an optionally substituted C₁-C₆ alkyl |
| | | alkyl & an unsubstituted C₁-C₆ | |
| | | haloalkyl | |
| 3 | 1 | —CN, an optionally substituted C₁-C₆ | an unsubstituted C₁-C₆ haloalkyl |
| | | alkyl & an unsubstituted C₁-C₆ | |
| | | haloalkyl | |
| 3 | 1 | —CN, an unsubstituted C₁-C₆ | halogen |
| | | haloalkyl & an unsubstituted C₁-C₆ | |
| | | haloalkyl | |
| 3 | 1 | —CN, an unsubstituted C₁-C₆ | an optionally substituted C₁-C₆ alkyl |
| | | haloalkyl & an unsubstituted C₁-C₆ | |
| | | haloalkyl | |
| 3 | 1 | —CN, an unsubstituted C₁-C₆ | an unsubstituted C₁-C₆ haloalkyl |
| | | haloalkyl & an unsubstituted C₁-C₆ | |
| | | haloalkyl | |
| 3 | 1 | —CN, halogen, & an optionally | halogen |
| | | substituted C₁-C₆ alkyl | |
| 3 | 1 | —CN, halogen, & an optionally | an optionally substituted C₁-C₆ alkyl |
| | | substituted C₁-C₆ alkyl | |
| 3 | 1 | —CN, halogen, & an optionally | an unsubstituted C₁-C₆ haloalkyl |
| | | substituted C₁-C₆ alkyl | |
| 3 | 1 | —CN, halogen, & an unsubstituted | halogen |
| | | C₁-C₆ haloalkyl | |
| 3 | 1 | —CN, halogen, & an unsubstituted | an optionally substituted C₁-C₆ alkyl |
| | | C₁-C₆ haloalkyl | |
| 3 | 1 | —CN, halogen, & an unsubstituted | an unsubstituted C₁-C₆ haloalkyl |
| | | C₁-C₆ haloalkyl | |
| 3 | 2 | halogen, halogen & halogen | halogen & halogen |
| 3 | 2 | halogen, halogen & halogen | halogen & an optionally substituted |
| | | | C₁-C₆ alkyl |
| 3 | 2 | halogen, halogen & halogen | halogen & an unsubstituted C₁-C₆ |
| | | | haloalkyl |
| 3 | 2 | halogen, halogen & halogen | an optionally substituted C₁-C₆ alkyl |
| | | | & an optionally substituted C₁-C₆ |
| | | | alkyl |
| 3 | 2 | halogen, halogen & halogen | an optionally substituted C₁-C₆ alkyl |
| | | | & an unsubstituted C₁-C₆ haloalkyl |
| 3 | 2 | halogen, halogen & halogen | an unsubstituted C₁-C₆ haloalkyl & an |
| | | | unsubstituted C₁-C₆ haloalkyl |
| 3 | 2 | halogen, halogen & an optionally | halogen & halogen |
| | | substituted C₁-C₆ alkyl | |
| 3 | 2 | halogen, halogen & an optionally | halogen & an optionally substituted |
| | | substituted C₁-C₆ alkyl | C₁-C₆ alkyl |
| 3 | 2 | halogen, halogen & an optionally | halogen & an unsubstituted C₁-C₆ |
| | | substituted C₁-C₆ alkyl | haloalkyl |
| 3 | 2 | halogen, halogen & an optionally | an optionally substituted C₁-C₆ alkyl |
| | | substituted C₁-C₆ alkyl | & an optionally substituted C₁-C₆ |
| | | | alkyl |
| 3 | 2 | halogen, halogen & an optionally | an optionally substituted C₁-C₆ alkyl |
| | | substituted C₁-C₆ alkyl | & an unsubstituted C₁-C₆ haloalkyl |
| 3 | 2 | halogen, halogen & an optionally | an unsubstituted C₁-C₆ haloalkyl & an |
| | | substituted C₁-C₆ alkyl | unsubstituted C₁-C₆ haloalkyl |
| 3 | 2 | halogen, halogen & an unsubstituted | halogen & halogen |
| | | C₁-C₆ haloalkyl | |
| 3 | 2 | halogen, halogen & an unsubstituted | halogen & an optionally substituted |
| | | C₁-C₆ haloalkyl | C₁-C₆ alkyl |
| 3 | 2 | halogen, halogen & an unsubstituted | halogen & an unsubstituted C₁-C₆ |
| | | C₁-C₆ haloalkyl | haloalkyl |
| 3 | 2 | halogen, halogen & an unsubstituted | an optionally substituted C₁-C₆ alkyl |
| | | C₁-C₆ haloalkyl | & an optionally substituted C₁-C₆ |
| | | | alkyl |
| 3 | 2 | halogen, halogen & an unsubstituted | an optionally substituted C₁-C₆ alkyl |
| | | C₁-C₆ haloalkyl | & an unsubstituted C₁-C₆ haloalkyl |
| 3 | 2 | halogen, halogen & an unsubstituted | an unsubstituted C₁-C₆ haloalkyl & an |
| | | C₁-C₆ haloalkyl | unsubstituted C₁-C₆ haloalkyl |
| 3 | 2 | halogen, an optionally substituted | halogen & halogen |
| | | C₁-C₆ alkyl & an optionally | |
| | | substituted C₁-C₆ alkyl | |
| 3 | 2 | halogen, an optionally substituted | halogen & an optionally substituted |
| | | C₁-C₆ alkyl & an optionally | C₁-C₆ alkyl |
| | | substituted C₁-C₆ alkyl | |
| 3 | 2 | halogen, an optionally substituted | halogen & an unsubstituted C₁-C₆ |
| | | C₁-C₆ alkyl & an optionally | haloalkyl |
| | | substituted C₁-C₆ alkyl | |
| 3 | 2 | halogen, an optionally substituted | an optionally substituted C₁-C₆ alkyl |
| | | C₁-C₆ alkyl & an optionally | & an optionally substituted C₁-C₆ |
| | | substituted C₁-C₆ alkyl | alkyl |
| 3 | 2 | halogen, an optionally substituted | an optionally substituted C₁-C₆ alkyl |
| | | C₁-C₆ alkyl & an optionally | & an unsubstituted C₁-C₆ haloalkyl |
| | | substituted C₁-C₆ alkyl | |
| 3 | 2 | halogen, an optionally substituted | an unsubstituted C₁-C₆ haloalkyl & an |
| | | C₁-C₆ alkyl & an optionally | unsubstituted C₁-C₆ haloalkyl |
| | | substituted C₁-C₆ alkyl | |
| 3 | 2 | halogen, an unsubstituted C₁-C₆ | halogen & halogen |
| | | haloalkyl & an unsubstituted C₁-C₆ | |
| | | haloalkyl | |
| 3 | 2 | halogen, an unsubstituted C₁-C₆ | halogen & an optionally substituted |
| | | haloalkyl & an unsubstituted C₁-C₆ | C₁-C₆ alkyl |
| | | haloalkyl | |
| 3 | 2 | halogen, an unsubstituted C₁-C₆ | halogen & an unsubstituted C₁-C₆ |
| | | haloalkyl & an unsubstituted C₁-C₆ | haloalkyl |
| | | haloalkyl | |
| 3 | 2 | halogen, an unsubstituted C₁-C₆ | an optionally substituted C₁-C₆ alkyl |
| | | haloalkyl & an unsubstituted C₁-C₆ | & an optionally substituted C₁-C₆ |
| | | haloalkyl | alkyl |
| 3 | 2 | halogen, an unsubstituted C₁-C₆ | an optionally substituted C₁-C₆ alkyl |
| | | haloalkyl & an unsubstituted C₁-C₆ | & an unsubstituted C₁-C₆ haloalkyl |
| | | haloalkyl | |
| 3 | 2 | halogen, an unsubstituted C₁-C₆ | an unsubstituted C₁-C₆ haloalkyl & an |
| | | haloalkyl & an unsubstituted C₁-C₆ | unsubstituted C₁-C₆ haloalkyl |
| | | haloalkyl | |
| 3 | 2 | an optionally substituted C₁-C₆ | halogen & halogen |
| | | alkyl, an optionally substituted C₁-C₆ alkyl | |
| | | & an unsubstituted C₁-C₆ | |
| | | haloalkyl | |
| 3 | 2 | an optionally substituted C₁-C₆ | halogen & an optionally substituted |
| | | alkyl, an optionally substituted C₁-C₆ alkyl | C₁-C₆ alkyl |
| | | & an unsubstituted C₁-C₆ | |
| | | haloalkyl | |
| 3 | 2 | an optionally substituted C₁-C₆ | halogen & an unsubstituted C₁-C₆ |
| | | alkyl, an optionally substituted C₁-C₆ alkyl | haloalkyl |
| | | & an unsubstituted C₁-C₆ | |
| | | haloalkyl | |
| 3 | 2 | an optionally substituted C₁-C₆ | an optionally substituted C₁-C₆ alkyl |
| | | alkyl, an optionally substituted C₁-C₆ alkyl | & an optionally substituted C₁-C₆ |
| | | & an unsubstituted C₁-C₆ | alkyl |
| | | haloalkyl | |
| 3 | 2 | an optionally substituted C₁-C₆ | an optionally substituted C₁-C₆ alkyl |
| | | alkyl, an optionally substituted C₁-C₆ alkyl | & an unsubstituted C₁-C₆ haloalkyl |
| | | & an unsubstituted C₁-C₆ | |
| | | haloalkyl | |
| 3 | 2 | an optionally substituted C₁-C₆ | an unsubstituted C₁-C₆ haloalkyl & an |
| | | alkyl, an optionally substituted C₁-C₆ alkyl | unsubstituted C₁-C₆ haloalkyl |
| | | & an unsubstituted C₁-C₆ | |
| | | haloalkyl | |
| 3 | 2 | an optionally substituted C₁-C₆ | halogen & halogen |
| | | alkyl, an unsubstituted C₁-C₆ | |
| | | haloalkyl & an unsubstituted C₁-C₆ | |
| | | haloalkyl | |
| 3 | 2 | an optionally substituted C₁-C₆ | halogen & an optionally substituted |
| | | alkyl, an unsubstituted C₁-C₆ | C₁-C₆ alkyl |
| | | haloalkyl & an unsubstituted C₁-C₆ | |
| | | haloalkyl | |
| 3 | 2 | an optionally substituted C₁-C₆ | halogen & an unsubstituted C₁-C₆ |
| | | alkyl, an unsubstituted C₁-C₆ | haloalkyl |
| | | haloalkyl & an unsubstituted C₁-C₆ | |
| | | haloalkyl | |
| 3 | 2 | an optionally substituted C₁-C₆ | an optionally substituted C₁-C₆ alkyl |
| | | alkyl, an unsubstituted C₁-C₆ | & an optionally substituted C₁-C₆ |
| | | haloalkyl & an unsubstituted C₁-C₆ | alkyl |
| | | haloalkyl | |
| 3 | 2 | an optionally substituted C₁-C₆ | an optionally substituted C₁-C₆ alkyl |
| | | alkyl, an unsubstituted C₁-C₆ | & an unsubstituted C₁-C₆ haloalkyl |
| | | haloalkyl & an unsubstituted C₁-C₆ | |
| | | haloalkyl | |
| 3 | 2 | an optionally substituted C₁-C₆ | an unsubstituted C₁-C₆ haloalkyl & an |
| | | alkyl, an unsubstituted C₁-C₆ | unsubstituted C₁-C₆ haloalkyl |
| | | haloalkyl & an unsubstituted C₁-C₆ | |
| | | haloalkyl | |
| 3 | 2 | halogen, an optionally substituted | halogen & halogen |
| | | C₁-C₆ alkyl & an unsubstituted | |
| | | C₁-C₆ haloalkyl | |
| 3 | 2 | halogen, an optionally substituted | halogen & an optionally substituted |
| | | C₁-C₆ alkyl & an unsubstituted | C₁-C₆ alkyl |
| | | C₁-C₆ haloalkyl | |
| 3 | 2 | halogen, an optionally substituted | halogen & an unsubstituted C₁-C₆ |
| | | C₁-C₆ alkyl & an unsubstituted | haloalkyl |
| | | C₁-C₆ haloalkyl | |
| 3 | 2 | halogen, an optionally substituted | an optionally substituted C₁-C₆ alkyl |
| | | C₁-C₆ alkyl & an unsubstituted | & an optionally substituted C₁-C₆ |
| | | C₁-C₆ haloalkyl | alkyl |
| 3 | 2 | halogen, an optionally substituted | an optionally substituted C₁-C₆ alkyl |
| | | C₁-C₆ alkyl & an unsubstituted | & an unsubstituted C₁-C₆ haloalkyl |
| | | C₁-C₆ haloalkyl | |
| 3 | 2 | halogen, an optionally substituted | an unsubstituted C₁-C₆ haloalkyl & an |
| | | C₁-C₆ alkyl & an unsubstituted | unsubstituted C₁-C₆ haloalkyl |
| | | C₁-C₆ haloalkyl | |
| 3 | 2 | —CN, halogen and halogen | halogen & halogen |
| 3 | 2 | —CN, halogen and halogen | halogen & an optionally substituted |
| | | | C₁-C₆ alkyl |
| 3 | 2 | —CN, halogen and halogen | halogen & an unsubstituted C₁-C₆ |
| | | | haloalkyl |
| 3 | 2 | —CN, halogen and halogen | an optionally substituted C₁-C₆ alkyl |
| | | | & an optionally substituted C₁-C₆ |
| | | | alkyl |
| 3 | 2 | —CN, halogen and halogen | an optionally substituted C₁-C₆ alkyl |
| | | | & an unsubstituted C₁-C₆ haloalkyl |
| 3 | 2 | —CN, halogen and halogen | an unsubstituted C₁-C₆ haloalkyl & an |
| | | | unsubstituted C₁-C₆ haloalkyl |
| 3 | 2 | —CN, an optionally substituted C₁-C₆ | halogen & halogen |
| | | alkyl & an optionally substituted C₁-C₆ alkyl | |
| 3 | 2 | —CN, an optionally substituted C₁-C₆ | halogen & an optionally substituted |
| | | alkyl & an optionally substituted C₁-C₆ alkyl | C₁-C₆ alkyl |
| 3 | 2 | —CN, an optionally substituted C₁-C₆ | halogen & an unsubstituted C₁-C₆ |
| | | alkyl & an optionally substituted C₁-C₆ alkyl | haloalkyl |
| 3 | 2 | —CN, an optionally substituted C₁-C₆ | an optionally substituted C₁-C₆ alkyl |
| | | alkyl & an optionally substituted C₁-C₆ alkyl | & an optionally substituted C₁-C₆ |
| | | | alkyl |
| 3 | 2 | —CN, an optionally substituted C₁-C₆ | an optionally substituted C₁-C₆ alkyl |
| | | alkyl & an optionally substituted C₁-C₆ alkyl | & an unsubstituted C₁-C₆ haloalkyl |
| 3 | 2 | —CN, an optionally substituted C₁-C₆ | an unsubstituted C₁-C₆ haloalkyl & an |
| | | alkyl & an optionally substituted C₁-C₆ alkyl | unsubstituted C₁-C₆ haloalkyl |
| 3 | 2 | —CN, an optionally substituted C₁-C₆ | halogen & halogen |
| | | alkyl & an unsubstituted C₁-C₆ | |
| | | haloalkyl | |
| 3 | 2 | —CN, an optionally substituted C₁-C₆ | halogen & an optionally substituted |
| | | alkyl & an unsubstituted C₁-C₆ | C₁-C₆ alkyl |
| | | haloalkyl | |
| 3 | 2 | —CN, an optionally substituted C₁-C₆ | halogen & an unsubstituted C₁-C₆ |
| | | alkyl & an unsubstituted C₁-C₆ | haloalkyl |
| | | haloalkyl | |
| 3 | 2 | —CN, an optionally substituted C₁-C₆ | an optionally substituted C₁-C₆ alkyl |
| | | alkyl & an unsubstituted C₁-C₆ | & an optionally substituted C₁-C₆ |
| | | haloalkyl | alkyl |
| 3 | 2 | —CN, an optionally substituted C₁-C₆ | an optionally substituted C₁-C₆ alkyl |
| | | alkyl & an unsubstituted C₁-C₆ | & an unsubstituted C₁-C₆ haloalkyl |
| | | haloalkyl | |
| 3 | 2 | —CN, an optionally substituted C₁-C₆ | an unsubstituted C₁-C₆ haloalkyl & an |
| | | alkyl & an unsubstituted C₁-C₆ | unsubstituted C₁-C₆ haloalkyl |
| | | haloalkyl | |
| 3 | 2 | —CN, an unsubstituted C₁-C₆ | halogen & halogen |
| | | haloalkyl & an unsubstituted C₁-C₆ | |
| | | haloalkyl | |
| 3 | 2 | —CN, an unsubstituted C₁-C₆ | halogen & an optionally substituted |
| | | haloalkyl & an unsubstituted C₁-C₆ | C₁-C₆ alkyl |
| | | haloalkyl | |
| 3 | 2 | —CN, an unsubstituted C₁-C₆ | halogen & an unsubstituted C₁-C₆ |
| | | haloalkyl & an unsubstituted C₁-C₆ | haloalkyl |
| | | haloalkyl | |
| 3 | 2 | —CN, an unsubstituted C₁-C₆ | an optionally substituted C₁-C₆ alkyl |
| | | haloalkyl & an unsubstituted C₁-C₆ | & an optionally substituted C₁-C₆ |
| | | haloalkyl | alkyl |
| 3 | 2 | —CN, an unsubstituted C₁-C₆ | an optionally substituted C₁-C₆ alkyl |
| | | haloalkyl & an unsubstituted C₁-C₆ | & an unsubstituted C₁-C₆ haloalkyl |
| | | haloalkyl | |
| 3 | 2 | —CN, an unsubstituted C₁-C₆ | an unsubstituted C₁-C₆ haloalkyl & an |
| | | haloalkyl & an unsubstituted C₁-C₆ | unsubstituted C₁-C₆ haloalkyl |
| | | haloalkyl | |
| 3 | 2 | —CN, halogen, & an optionally | halogen & halogen |
| | | substituted C₁-C₆ alkyl | |
| 3 | 2 | —CN, halogen, & an optionally | halogen & an optionally substituted |
| | | substituted C₁-C₆ alkyl | C₁-C₆ alkyl |
| 3 | 2 | —CN, halogen, & an optionally | halogen & an unsubstituted C₁-C₆ |
| | | substituted C₁-C₆ alkyl | haloalkyl |
| 3 | 2 | —CN, halogen, & an unsubstituted | an optionally substituted C₁-C₆ alkyl |
| | | C₁-C₆ haloalkyl | & an optionally substituted C₁-C₆ |
| | | | alkyl |
| 3 | 2 | —CN, halogen, & an unsubstituted | an optionally substituted C₁-C₆ alkyl |
| | | C₁-C₆ haloalkyl | & an unsubstituted C₁-C₆ haloalkyl |
| 3 | 2 | —CN, halogen, & an unsubstituted | an unsubstituted C₁-C₆ haloalkyl & an |
| | | C₁-C₆ haloalkyl | unsubstituted C₁-C₆ haloalkyl |

In any of the embodiment in Table 2, each halogen can be independently fluoro or chloro. In any of the embodiments in Table 2, each optionally substituted C₁-C₆ alkyl can be independently a substituted C₁-C₆ alkyl. In any of the embodiments in Table 2, each optionally substituted C₁-C₆ alkyl can be independently an unsubstituted C₁-C₆ alkyl. Examples of C₁-C₆ alkyl groups are provided herein. In any of the embodiments in Table 2, each optionally substituted C₃-C₆ cycloalkyl can be independently a substituted C₃-C₆ cycloalkyl. In any of the embodiments in Table 2, each optionally substituted C₃-C₆ cycloalkyl can be independently an unsubstituted C₃-C₆ cycloalkyl. Examples of C₃-C₆ cycloalkyl groups are provided herein. In any of the embodiments in Table 2, the unsubstituted C₁-C₆ haloalkyl can be selected from —CF₃, —CCl₃, —CHF₂, —CHCl₂, —CH₂F, —CH₂Cl, —CH₂CF₃, —CF₂CHF₂, —CF₂CF₃and —CF₂Cl. In any of the embodiments in Table 2, R³ can be an optionally substituted C₁-C₆ alkyl. In any of the embodiments in Table 2, R³ can be a substituted C₁-C₆ alkyl. In any of the embodiments in Table 2, R³ can be an unsubstituted C₁-C₆ alkyl. Examples of C₁-C₆ alkyl groups are provided herein. In any of the embodiments in Table 2, R³ can be ethyl. In any of the embodiments in Table 2, R³ can be an unsubstituted C₁-C₆ haloalkyl, such as —CF₃, —CCl₃, —CHF₂, —CHCl₂, —CH₂F, —CH₂Cl, —CH₂CF₃, —CF₂CHF₂, —CF₂CF₃and —CF₂Cl. In any of the embodiments in Table 2, R³ can be an optionally substituted C₃-C₆ cycloalkyl. In any of the embodiments in Table 2, R³ can be a substituted C₃-C₆ cycloalkyl. In any of the embodiments in Table 2, R³ can be an unsubstituted C₃-C₆ cycloalkyl. Examples of C₃-C₆ cycloalkyl groups are provided herein.

In some embodiments, when n is 0, then m is 1, 2, 3, 4 or 5. In some embodiments, m and n are not both 0.

In some embodiments, when m is 1 or 2, at least one R¹ substituent can be at the ortho-position to the bond connecting the phenyl ring to the rest of the molecule. In other embodiments, when m is 1 or 2, at least one R¹ substituent can be at the meta-position to the bond connecting the phenyl ring to the rest of the molecule. In still other embodiments, when m is 1 or 2, at least one R¹ substituent can be at the para-position to the bond connecting the phenyl ring to the rest of the molecule. In some embodiments, when m is 2, one R¹ substituent can be at the ortho-position and the other R¹ can be at the para-position relative to the point of attachment of the phenyl ring to the rest of the compound of Formula (I). In some embodiments, when m is 2 the R¹ substituents can be attached to adjacent rings carbon (the R¹ substituents occupy positions that are ortho to each other). In other embodiments, when m is 2 the R¹ substituents can be separated by 1 ring carbon (the R¹ substituents occupy positions that are meta to each other). In still other embodiments, when m is 2 the R¹ can be separated by 2 ring carbons (the R¹ substituents occupy positions that are para to each other).

In some embodiments, when n is 2 the R² substituents can be attached to adjacent ring carbons (the R² substituents occupy positions that are ortho to each other). In other embodiments, when n is 2 the R² can be separated by 2 ring carbons (the R¹ substituents occupy positions that are para to each other). In still other embodiments, when n is 2 the R² substituents can be separated by 1 ring carbon (the R² substituents occupy positions that are meta to each other).

Examples of a compound of Formula (I) include:

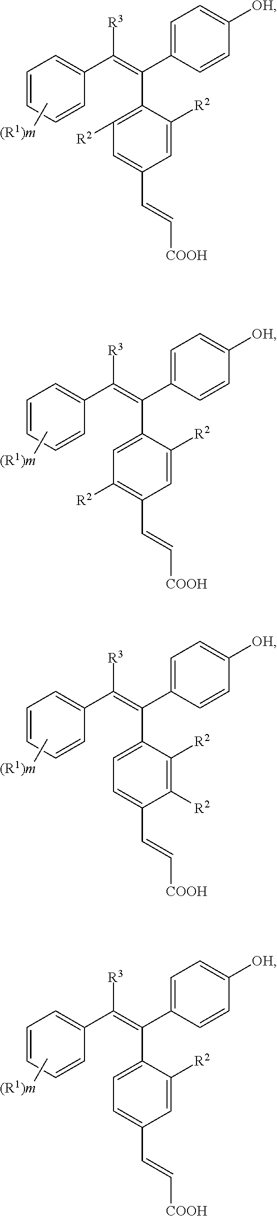

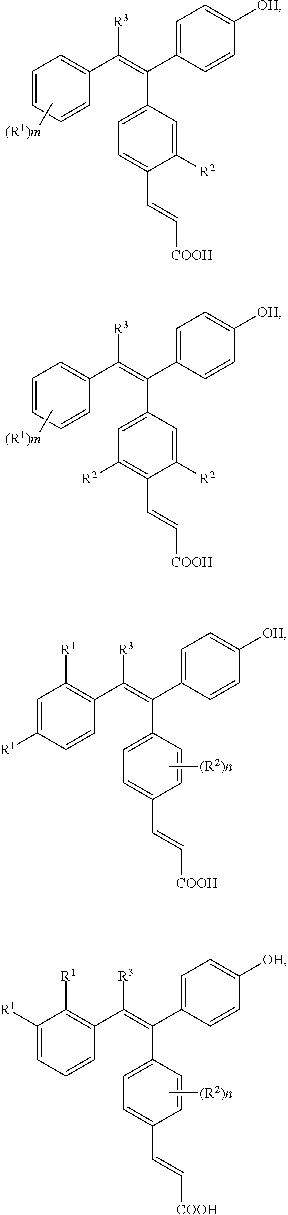

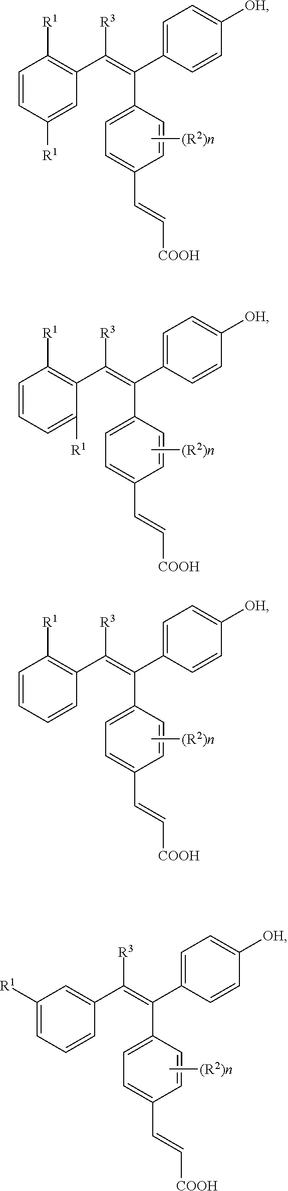

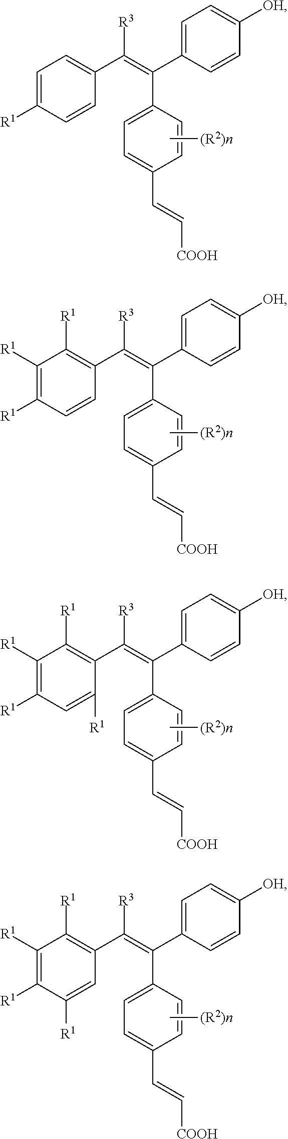

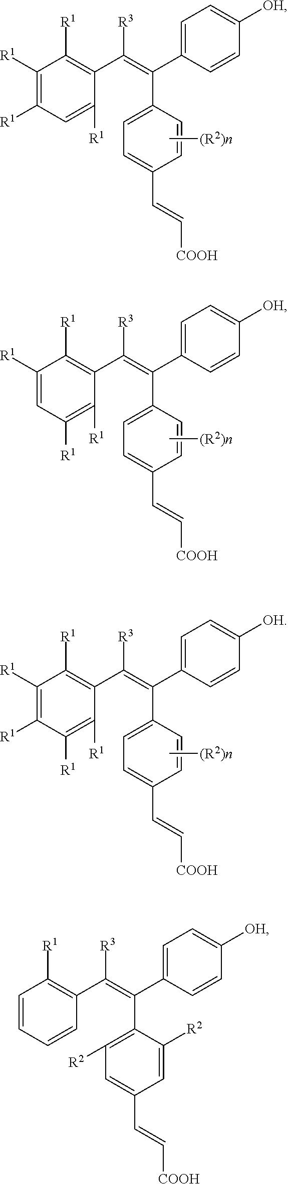

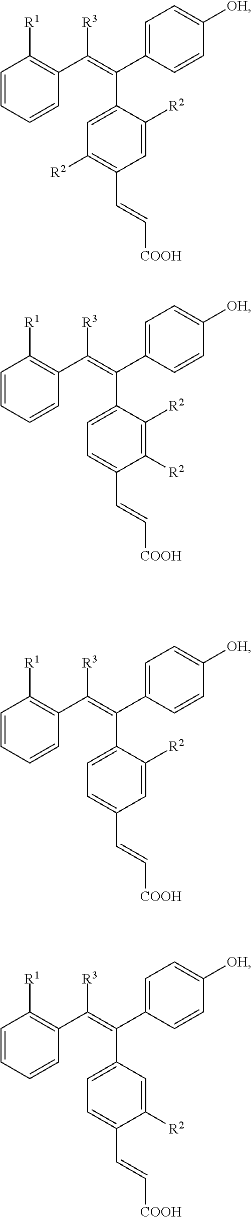

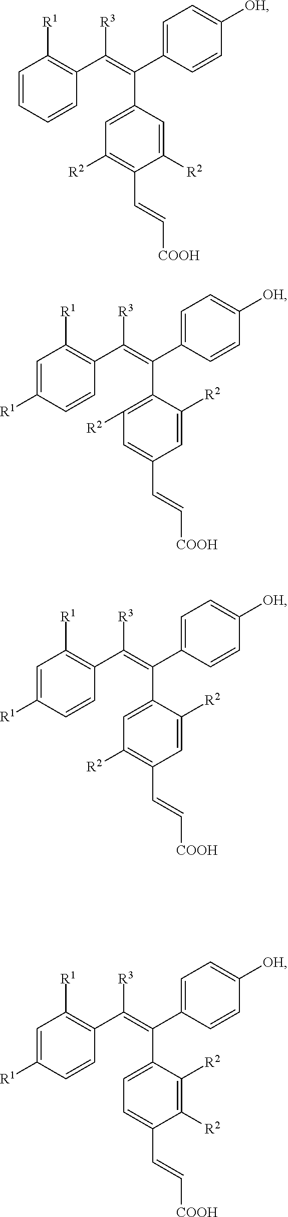

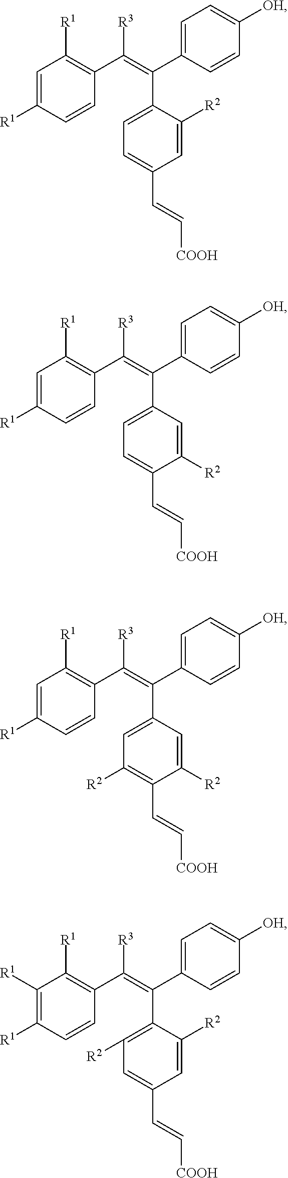

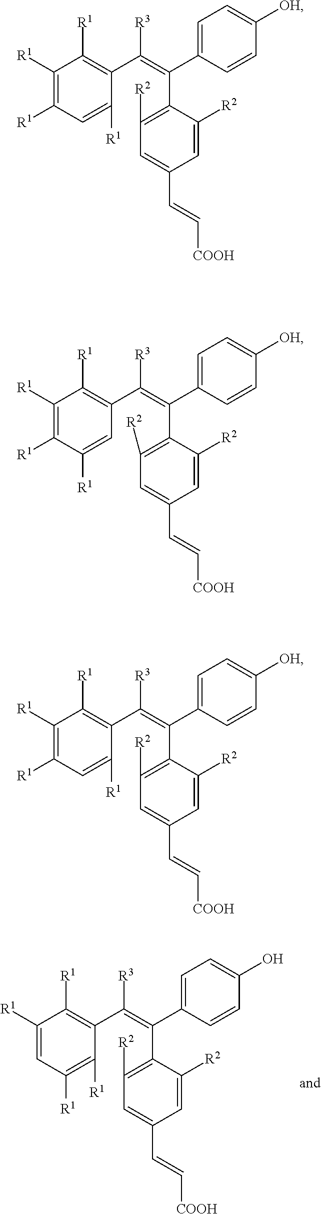

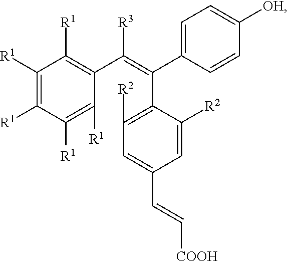

or a pharmaceutically acceptable salt of any of the foregoing, wherein R¹, R², R³, m and n are as defined herein.

Examples of compound of Formula (I) include the following:

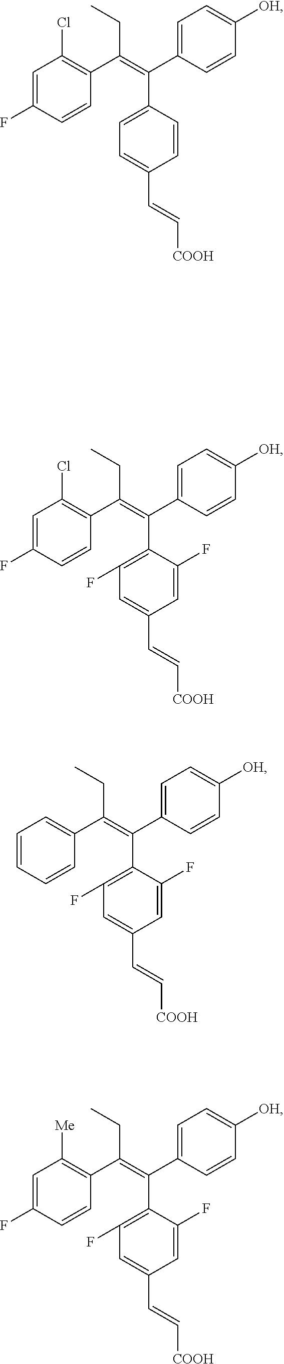 [SMILES: CC/C(=C(\c1ccc(O)cc1)c1ccc(/C=C/C(=O)O)cc1)c1ccc(F)cc1Cl ; CC/C(=C(\c1ccc(O)cc1)c1c(F)cc(/C=C/C(=O)O)cc1F)c1ccc(F)cc1Cl ; CC/C(=C(\c1ccc(O)cc1)c1c(F)cc(/C=C/C(=O)O)cc1F)c1ccccc1 ; CC/C(=C(\c1ccc(O)cc1)c1c(F)cc(/C=C/C(=O)O)cc1F)c1ccc(F)cc1C]

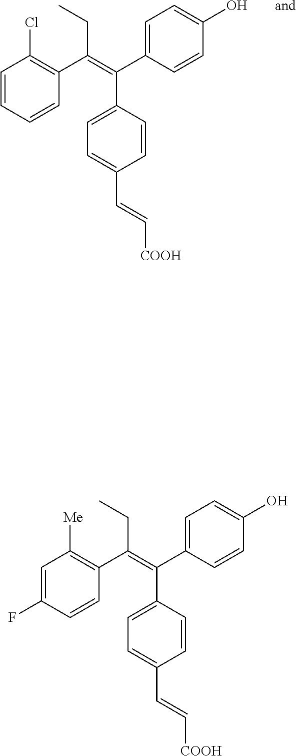 [SMILES: CC/C(=C(\c1ccc(O)cc1)c1ccc(/C=C/C(=O)O)cc1)c1ccccc1Cl ; "and" ; CC/C(=C(\c1ccc(O)cc1)c1ccc(/C=C/C(=O)O)cc1)c1ccc(F)cc1C]

or a pharmaceutically acceptable salt of any of the foregoing.

### Synthesis

Compounds of the Formula (I), or pharmaceutically acceptable salts thereof, can be made in various ways by those skilled using known techniques as guided by the detailed teachings provided herein. For example, in an embodiment, compounds of the Formula (I) are prepared in accordance with General Scheme 1 as shown herein.

One method for forming a compound of Formula (I) is shown in the general scheme below, wherein R¹, R², R³, m and n are described herein. In general, the coupling reactions, deprotection reactions and olefination reactions between compounds of the general Formulae C, D, E, F and G to form compounds of the Formula (I) as illustrated in General Scheme 1 can be carried out in a manner similar to the reactions as described herein in Example 1. Any preliminary reaction steps required to form starting compounds of the general Formula A, or other precursors, can be carried out by those skilled in the art. Similarly, any intermediate reaction products formed as a result of the reactions providing compounds of the general Formulae B, C, D, E, F and G can be readily converted to compounds of the Formula (I) by those skilled in the art in view of the detailed teaching provided herein, for example, by appropriate adjustment of the reagents and conditions described in Example 1 for the preparation of (E)-3-(4-((E)-2-(2-chloro-4-fluorophenyl)-1-(4-hydroxyphenyl)but-1-en-1-yl)phenyl)acrylic acid. In some embodiments, the R¹ and R² group(s) can be introduced by successive palladium-catalyzed Suzuki couplings using R¹- or R²-functionalized aryl groups. The remaining transformations are carried out as described herein, for example, in Example 1.

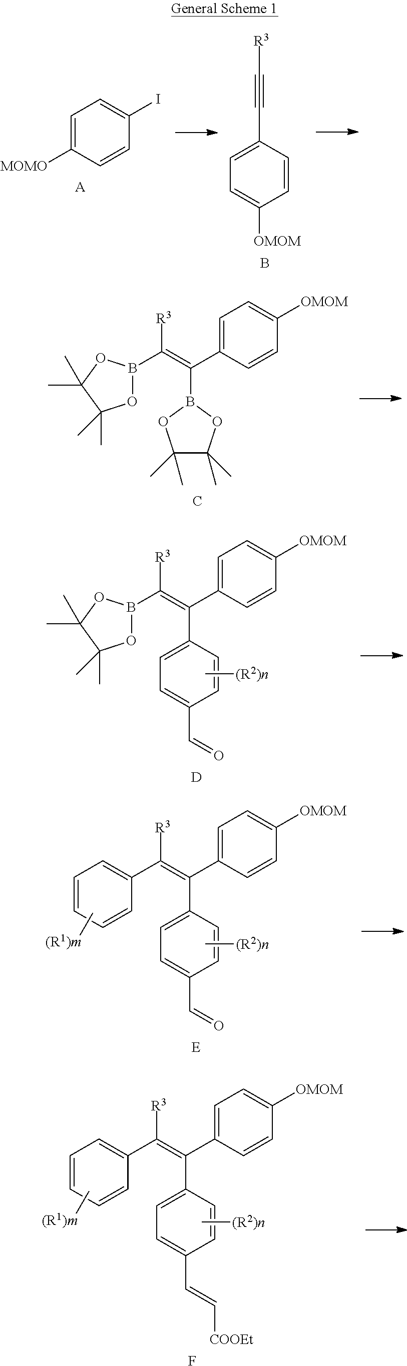

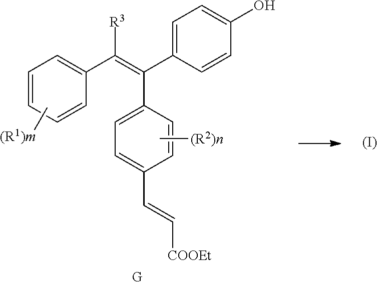

### Pharmaceutical Compositions

Some embodiments described herein relate to a pharmaceutical composition, that can include an effective amount of one or more compounds described herein (for example, a compound of Formula (I), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

Some embodiments disclosed herein relate to a method of treatment that can include identifying a subject that is in need of treatment for a disease or condition that is ER alpha dependent, and/or ER alpha mediated; and administering to said subject an effective amount of a compound of Formula (I), or a pharmaceutically acceptable salt thereof, or a pharmaceutical composition that can include a compound of Formula (I), or a pharmaceutically acceptable salt thereof. Other embodiments disclosed herein relate to the use of a compound of Formula (I), or a pharmaceutically acceptable salt thereof, or a pharmaceutical composition that can include a compound of Formula (I), or a pharmaceutically acceptable salt thereof, in the preparation of a medicament for the treatment of a disease or condition that is ER alpha dependent, and/or ER alpha mediated. Still other embodiments disclosed herein relate to a compound of Formula (I), or a pharmaceutically acceptable salt thereof, or a pharmaceutical composition that can include a compound of Formula (I), or a pharmaceutically acceptable salt thereof, for use in the treatment of a disease or condition that is ER alpha dependent, and/or ER alpha mediated.

In various embodiments, compounds of Formula (I), or a pharmaceutically acceptable salt thereof, can be useful for ameliorating or treating a disease or condition that is ER dependent and/or ER mediated. In some embodiments, the disease or condition is ER alpha dependent and/or ER alpha mediated. In some embodiments, the disease can be a cancer. In some embodiments, the cancer can be a lung cancer (for example, non-small cell lung cancer and small cell lung cancer), a breast and/or a gynecological cancer. In some embodiments, the cancer can be selected from a lung cancer (for example, non-small cell lung cancer and small cell lung cancer), breast cancer, endometrial cancer, ovarian cancer and cervical cancer. In some embodiments, the cancer can be selected from a breast cancer, an endometrial cancer, an ovarian cancer and a cervical cancer. In some embodiments, the cancer can be a breast cancer. In some embodiments, the cancer can be a lung cancer (for example, non-small cell lung cancer and small cell lung cancer). In some embodiments, compounds of Formula (I), or a pharmaceutically acceptable salt thereof, can be selective ER modulators (SERMs) and/or can be selective ER degraders (SERDs). In some embodiments, compounds of Formula (I), or a pharmaceutically acceptable salt thereof, can be a SERM. In some embodiments, compounds of Formula (I), or a pharmaceutically acceptable salt thereof, can be a SERD. Additional details regarding various uses and methods of treatment are described elsewhere herein.

Some embodiments disclosed herein relate a method of inhibiting the growth of a cell, that can include identifying a cell having an ER alpha that mediates a growth characteristic of the cell; and contacting the cell with an effective amount of the compound of Formula (I), or a pharmaceutically acceptable salt thereof. Other embodiments disclosed herein relate to the use of a compound of Formula (I), or a pharmaceutically acceptable salt thereof, or a pharmaceutical composition that can include a compound of Formula (I), or a pharmaceutically acceptable salt thereof, in the preparation of a medicament for inhibiting the growth of a cell, that has an ER alpha that mediates a growth characteristic of the cell. Still other embodiments disclosed herein relate to a compound of Formula (I), or a pharmaceutically acceptable salt thereof, or a pharmaceutical composition that can include a compound of Formula (I), or a pharmaceutically acceptable salt thereof, for use in inhibiting the growth of a cell that has an ER alpha that mediates a growth characteristic of the cell.

As described herein, one or more compounds of Formula (I), or pharmaceutically acceptable salts thereof, or a pharmaceutical composition as described herein, can be used to inhibit the growth of a cell. In some embodiments, the cell can be identified as having an ER that mediates a growth characteristic of the cell. Growth of a cell can be inhibited by contacting the cell with an effective amount of at least one of the compounds described herein, or pharmaceutically acceptable salts thereof, or a pharmaceutical composition as described elsewhere herein.

Such contacting of the one or more compounds, or pharmaceutically acceptable salts thereof, can take place in various ways and locations, including without limitation away from a living subject (e.g., in a laboratory, diagnostic and/or analytical setting) or in proximity to a living subject (e.g., within or on an exterior portion of an animal, e.g., a human) For example, an embodiment provides a method of treating a subject, that can include identifying a subject that is in need of treatment for a disease or condition that is ER alpha dependent and/or ER alpha mediated and administering to said subject an effective amount of one or more compounds of Formula (I), or a pharmaceutically acceptable salt thereof, or a pharmaceutical composition, as described elsewhere herein. In some embodiments, the ER is ER alpha. In some embodiments, the subject is known to possess wild-type ER alpha. In some embodiments, the subject is known to overexpress ER alpha. In some embodiments, the subject is known to possess mutant ER alpha. In some embodiments, the subject is known to possess mutant ER alpha with one or more mutations selected from E380Q, S463P, P535H, L536Q, L536R, L536H, L536P, Y537C, Y537N, Y537S, D538G and V543E. Additional embodiments provide a method of treatment that can include administering an effective amount of one or more compounds of Formula (I), or a pharmaceutically acceptable salt thereof, or a pharmaceutical composition, as described elsewhere herein, to a subject having a disease or condition that is ER alpha dependent and/or ER alpha mediated.

As described herein, compounds of Formula (I), or a pharmaceutically acceptable salt thereof, or a pharmaceutical composition as described elsewhere herein, can be administered to such subjects by a variety of methods. In any of the methods described herein, administration can be by various routes known to those skilled in the art, including without limitation oral, intravenous, intramuscular, topical, subcutaneous, systemic and/or intraperitoneal administration to a subject in need thereof.

As used herein, a “subject” refers to an animal that is the object of treatment, observation or experiment. “Animal” includes cold- and warm-blooded vertebrates and invertebrates such as fish, shellfish, reptiles and, in particular, mammals “Mammal” includes, without limitation, mice, rats, rabbits, guinea pigs, dogs, cats, sheep, goats, cows, horses, primates, such as monkeys, chimpanzees, and apes, and, in particular, humans In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the ER dependent and/or ER mediated disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the ER dependent and/or ER mediated disease or condition, or prolong the survival of the subject being treated. This response may occur in a tissue, system, animal or human and includes alleviation of the signs or symptoms of the ER dependent and/or ER mediated disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

The amount of the compound of Formula (I), or a pharmaceutically acceptable salt thereof, required for use in treatment will vary not only with the particular compound or salt selected but also with the route of administration, the nature and/or symptoms of the ER dependent and/or ER mediated disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive ER dependent and/or ER mediated diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formula (I), or pharmaceutically acceptable salts thereof, can be determined by comparing their in vitro activity, and in vivo activity in animal models. Such comparison can be done by comparison against an established drug, such as fulvestrant.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the ER dependent and/or ER mediated disease or condition to be treated and to the route of administration. The severity of the ER dependent and/or ER mediated disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Example 1

### (E)-3-(4-((E)-2-(2-chloro-4-fluorophenyl)-1-(4-hydroxyphenyl)but-1-en-1-yl)phenyl)acrylic acid

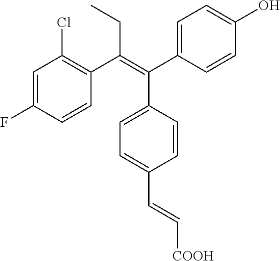 [SMILES: CC/C(=C(\c1ccc(O)cc1)c1ccc(/C=C/C(=O)O)cc1)c1ccc(F)cc1Cl]

To a sealed tube were added Pd(OAc)₂ (2.54 g, 3.79 mmol), 1,1′-bis(diphenylphosphanyl) ferrocene (2.09 g, 3.78 mmol), Cs₂CO₃ (31.8 g, 97 7 mmol), KF (0.87 g, 15 2 mmol) and a degassed solution of 1-iodo-4-(methoxymethoxy)benzene (20 g, 75.8 mmol) in N,N-dimethylacetamide (200 mL) at room temperature (rt). The mixture was stirred for 10 min and then bubbled with argon, followed by the addition of but-1-ynyltrimethylsilane (28.6 g, 227 mmol) and Cul (0.71 g, 3.79 mmol). After being stirred at 100° C. for 16 h, the mixture was cooled to rt, diluted with water (50 mL), and extracted with ethyl acetate (EtOAc)(50 mL). The organic phase was separated, dried over Na₂SO₄, filtered and concentrated under reduced pressure. The residue was purified by chromatography (SiO₂, 10% EtOAc/hexane) to afford 1-(but-1-ynyl)-4-(methoxymethoxy)benzene as a pale brown liquid (10.0 g, 69%). ¹H NMR (300 MHz, CDCl₃) δ 7.31 (d, J=9.0 Hz, 2H), 6.93 (d, J=9.0 Hz, 2H), 5.16 (s, 2H), 3.46 (s, 3H), 2.41 (q, J=6.8 Hz, 2H), 1.27 (t, J=6.9 Hz, 3H).

To a stirred solution of 1-(but-1-yn-1-yl)-4-(methoxymethoxy)benzene (10 g, 52.6 mmol) in 2-methyltetrahydrofuran (100 mL) was added 4,4,4′,4′,5,5,5′,5′-octamethyl-2,2′-bi(1,3,2-dioxaborolane) (14.7 g, 57.9 mmol). Argon was bubbled through the mixture for 10 min followed by the addition of Pt(PPh₃)4 (0.69 g, 0.52 mmol). After being stirred at 60° C. for 12 h, the mixture was cooled to rt, diluted with water (50 mL) and extracted with ethyl acetate (50 mL). The organic layer was separated, washed with water and brine, dried over Na₂SO₄, filtered and concentrated under reduced pressure. The residue was purified by chromatography (SiO₂, 10% EtOAc/hexane) to afford 2,2′-(1-(4-(methoxymethoxy)phenyl)but-1-ene-1,2-diyl)bis(4,4,5,5-tetramethyl-1,3,2-dioxaborolane) as a yellow liquid (8.0 g, 34%). ¹H NMR (300 MHz, CDCl₃) δ 7.02 (d, J=9 Hz, 2H), 6.92 (d, J=9 Hz, 2H), 5.16 (s, 2H), 3.49 (s, 3H), 2.11 (q, J=7.2 Hz, 2H), 1.33 (s, 12H), 1.27 (s, 12H), 0.95 (t, J=6.2 Hz, 3H).

To a stirred solution of 2,2′-(1-(4-(methoxymethoxy)phenyl)but-1-ene-1,2-diyl)bis(4,4,5,5-tetramethyl-1,3,2-dioxaborolane) (3.1 g, 6.98 mmol) in 2-methyltetrahydrofuran (30 mL) were added 4-iodobenzaldehyde (1.93 g, 8.37 mmol) and Cs₂CO₃ (5.68 g, 17.5 mmol). Argon was bubbled through the mixture for 10 min followed by the addition of PdCl₂(PPh₃)₂ (0.48 g, 0.698 mmol). After being stirred at 40° C. for 12 h, the mixture was cooled to rt and quenched with water/EtOAc (1:1, total of 100 mL). The organic layer was separated, dried over Na₂SO₄, filtered and concentrated under reduced pressure. The residue was purified by chromatography (SiO₂, 10% EtOAc/hexane) to afford (Z)-4-(1-(4-(methoxymethoxy)phenyl)-2-(4,4,5,5-tetramethyl-1,3,2-dioxaborolan-2-yl)but-1-en-1-yl)benzaldehyde as a yellow liquid (2.0 g, 69% with ˜10-15% of another regioisomer). Major isomer: ¹H NMR (300 MHz, CDCl₃) δ 9.97 (s, 1H), 7.75 (d, J=9.0 Hz, 2H), 7.36-7.34 (m, 2H), 7.05-6.96 (m, 4H), 5.16 (s, 2H), 3.48 (s, 3H), 2.31 (q, J=6.7 Hz, 2H), 1.25 (t, J=6.9 Hz, 3H), 1.10 (s, 12H); MS (ESI) m/z 423.21 [M+H]⁺.

To a stirred solution of (Z)-4-(1-(4-(methoxymethoxy)phenyl)-2-(4,4,5,5-tetramethyl-1,3,2-dioxaborolan-2-yl)but-1-en-1-yl)benzaldehyde (12 g, 28 4 mmol, with ˜10-15% of another regioisomer) in 2-methyltetrahydrofuran (120 mL) were added 2-chloro-4-fluoro-1-iodobenzene (5.5 mL, 40.5 mmol) and KOH (45 mL, 3M aqueous solution, 142.2 mmol). The resultant mixture was degassed with argon for 10 min followed by the addition of PdCl₂(PPh₃)₂ (0.7 g, 2.84 mmol). After stiffing at 60° C. for 12 h, the mixture was cooled to rt and quenched with water:EtOAc (1:1, 100 mL). The organic layer was separated, washed with brine, dried over Na₂SO₄, filtered and concentrated under reduced pressure. The crude compound was purified by chromatography (SiO₂, 10% EtOAc/hexane) to afford (E)-4-(2-(2-chloro-4-fluorophenyl)-1-(4-(methoxymethoxy) phenyl) but-1-en-1-yl)benzaldehyde as a pale yellow solid (6.0 g, 67% with ˜10-15% of the other regioisomer). Major isomer: ¹H NMR (400 MHz, CDCl₃) δ 9.84 (s, 1H), 7.55 (d, J=8.0 Hz, 2H), 7.20-7.17 (m, 2H), 7.10 (d, J=8.0 Hz, 2H), 7.04-6.97 (m, 4H), 6.84-6.80 (m, 1H), 5.19 (s, 2H), 3.50 (s, 3H), 2.55-2.45 (m, 2H), 0.97-0.93 (m, 3H); MS (ESI) m/z 425.19 [M+H]⁺.

To a stirred solution of (E)-4-(2-(2-chloro-4-fluorophenyl)-1-(4-(methoxymethoxy)phenyl)but-1-en-1-yl)benzaldehyde (5.3 g, 12 3 mmol, with ˜10-15% of the other regioisomer) in ACN (60 mL) was added ethyl 2-(diethoxyphosphoryl)acetate (3.36 g, 15 mmol), LiCl (1.05 g, 25 mmol) and DBU (2 mL, 13.8 mmol) at rt. After stiffing at rt for 16 h, the mixture was quenched with water/EtOAc (1:1, total of 100 mL). The organic layer was separated, washed with brine, dried over Na₂SO₄, filtered and concentrated under reduced pressure. The residue was purified by chromatography (SiO₂, 10% EtOAc/hexane) to afford ethyl (E)-3-(4-((E)-2-(2-chloro-4-fluorophenyl)-1-(4-(methoxymethoxy)phenyl)but-1-en-1-yl)phenyl)acrylate (5 g, 82%, off-white solid, with ˜10-15% of the other regioisomer). MS (ESI): m/z 495.18 [M+H]⁺.

To a stirred solution of ethyl (E)-3-(4-((E)-2-(2-chloro-4-fluorophenyl)-1-(4-(methoxymethoxy)phenyl)but-1-en-1-yl)phenyl)acrylate (5 g, 10.1 mmol, with ˜10-15% of the other regioisomer) in MeOH (50 mL) was added PTSA (1.35 g, 7.08 mmol) at rt. After stiffing at rt for 16 h, the mixture was concentrated and the residue purified by chromatography (SiO₂, 25% EtOAc/hexane) to afford ethyl (E)-3-(4-((E)-2-(2-chloro-4-fluorophenyl)-1-(4-hydroxyphenyl)but-1-en-1-yl)phenyl)acrylate as an off-white solid (4.1 g, 91% (with ˜10-15% of the other regioisomer)). MS (ESI) m/z 451.21 [M+H]⁺.

To a stirred solution of ethyl (E)-3-(4-((E)-2-(2-chloro-4-fluorophenyl)-1-(4-hydroxyphenyl)but-1-en-1-yl)phenyl)acrylate (5.2 g, 9.45 mmol, with ˜10-15% of the other regioisomer) in THF:water (2:1, 60 mL) was added LiOH (1.3 g, 56.7 mmol) at rt. After stiffing at rt for 16 h, the mixture was extracted with ethyl acetate and the aqueous layer was acidified with an aqueous solution of HCl (1N) to form a precipitate. The precipitate was filtered and purified by chromatography (SiO₂, 30% EtOAc/hexane) to afford 4.3 g of (E)-3-(4-((E)-2-(2-chloro-4-fluorophenyl)-1-(4-hydroxyphenyl)but-1-en-1-yl)phenyl) acrylic acid, with ˜10-15% of the other regioisomer. Further purification by rpHPLC afforded (E)-3-(4-((E)-2-(2-chloro-4-fluorophenyl)-1-(4-hydroxyphenyl)but-1-en-1-yl)phenyl)acrylic acid (1) after lyophilization of desired fractions (1.4 g, 35%). ¹H NMR (400 MHz, DMSO-d6) δ 9.50 (brs, 2H), 7.35-7.27 (m, 5H), 7.12-7.07 (m, 1H), 7.04 (d, J=8.4 Hz, 2H), 6.90 (d, J=8.0 Hz, 2H), 6.80 (d, J=8.0 Hz, 2H), 6.36 (d, J=16.0 Hz, 1H), 2.40 (q, J=8.0 Hz, 2H), 0.87 (t, J=8 Hz, 3H); MS (ESI) m/z 423.18 [M+H]⁺.

The compounds of Formula (I) illustrated in Table 3 can be prepared in various ways, using techniques known to those skilled in the art as guided by the detailed teachings provided herein. For example, the compounds of Formula (I) illustrated in Table 3 can be prepared in accordance with General Scheme 1 as described below.

TABLE 3

| No. | Compound Structure |
|---|---|
| 2 | 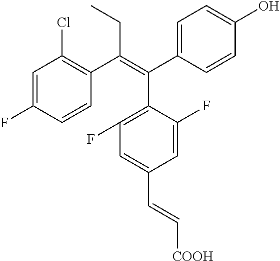 [SMILES: CC/C(=C(\c1ccc(O)cc1)c1c(F)cc(/C=C/C(=O)O)cc1F)c1ccc(F)cc1Cl] |
| 3 | 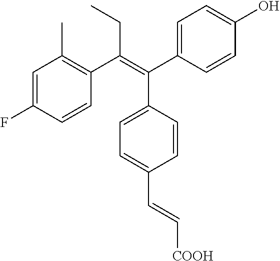 [SMILES: CC/C(=C(\c1ccc(O)cc1)c1ccc(/C=C/C(=O)O)cc1)c1ccc(F)cc1C] |
| 4 | 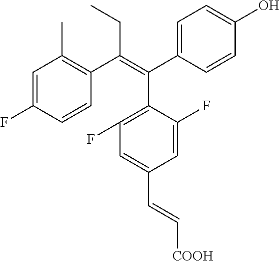 [SMILES: CC/C(=C(\c1ccc(O)cc1)c1c(F)cc(/C=C/C(=O)O)cc1F)c1ccc(F)cc1C] |
| 5 | 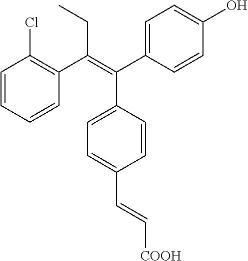 [SMILES: CC/C(=C(\c1ccc(O)cc1)c1ccc(/C=C/C(=O)O)cc1)c1ccccc1Cl] |
| 6 | 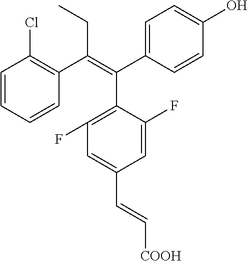 [SMILES: CC/C(=C(\c1ccc(O)cc1)c1c(F)cc(/C=C/C(=O)O)cc1F)c1ccccc1Cl] |
| 7 | 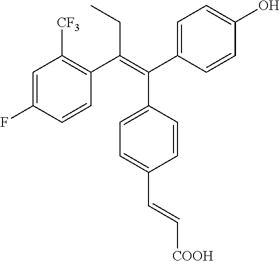 [SMILES: CC/C(=C(\c1ccc(O)cc1)c1ccc(/C=C/C(=O)O)cc1)c1ccc(F)cc1C(F)(F)F] |
| 8 | 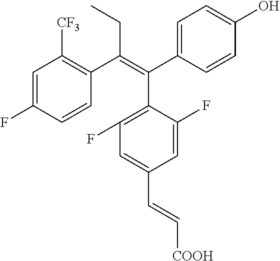 [SMILES: CC/C(=C(\c1ccc(O)cc1)c1c(F)cc(/C=C/C(=O)O)cc1F)c1ccc(F)cc1C(F)(F)F] |
| 9 | 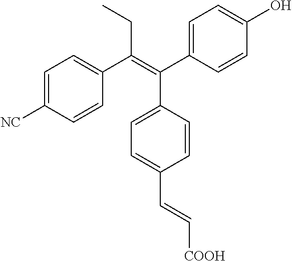 [SMILES: CC/C(=C(\c1ccc(O)cc1)c1ccc(/C=C/C(=O)O)cc1)c1ccc(C#N)cc1] |
| 10 | 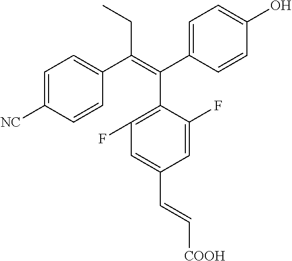 [SMILES: CC/C(=C(\c1ccc(O)cc1)c1c(F)cc(/C=C/C(=O)O)cc1F)c1ccc(C#N)cc1] |
| 11 | 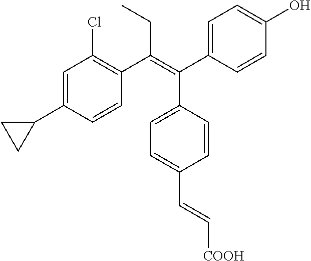 [SMILES: CC/C(=C(\c1ccc(O)cc1)c1ccc(/C=C/C(=O)O)cc1)c1ccc(C2CC2)cc1Cl] |
| 12 | 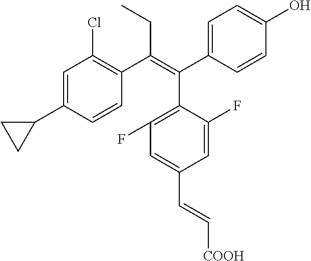 [SMILES: CC/C(=C(\c1ccc(O)cc1)c1c(F)cc(/C=C/C(=O)O)cc1F)c1ccc(C2CC2)cc1Cl] |
| 13 | 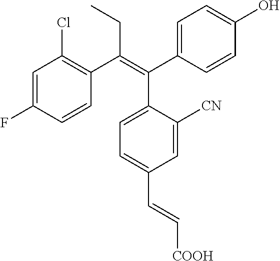 [SMILES: CC/C(=C(\c1ccc(O)cc1)c1ccc(/C=C/C(=O)O)cc1C#N)c1ccc(F)cc1Cl] |
| 14 | 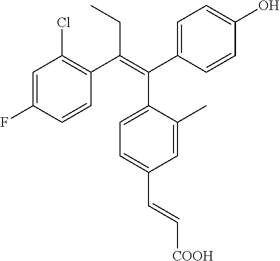 [SMILES: CC/C(=C(\c1ccc(O)cc1)c1ccc(/C=C/C(=O)O)cc1C)c1ccc(F)cc1Cl] |
| 15 | 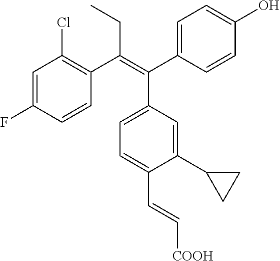 [SMILES: CC/C(=C(\c1ccc(O)cc1)c1ccc(/C=C/C(=O)O)c(C2CC2)c1)c1ccc(F)cc1Cl] |
| 16 | 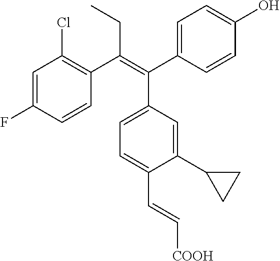 [SMILES: CC/C(=C(\c1ccc(O)cc1)c1ccc(/C=C/C(=O)O)c(C2CC2)c1)c1ccc(F)cc1Cl] |
| 17 | 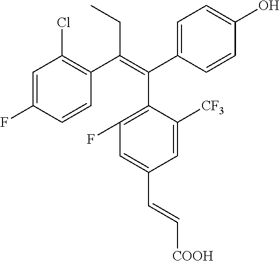 [SMILES: CC/C(=C(\c1ccc(O)cc1)c1c(F)cc(/C=C/C(=O)O)cc1C(F)(F)F)c1ccc(F)cc1Cl] |

### Example A

### Breast Cancer Cell Proliferation Assay (MCF-7)

MCF7 cells were expanded and maintained in DMEM (Hyclone SH30272.01, Phenol red free) with NEAA (Gibco11140-050), Na-pyruvate (Gibco 11360-070) and Re-stripped Charcoal stripped FBS (Gemini 100-119)). The cells were adjusted to a concentration of 3,000 cells per mL and incubated (37° C., 5% CO₂). The following day a 10 point, serial dilution of compounds was added to the cells at a final concentration ranging from 10 μM to 0.000005 μM for test compounds (17β-estradiol was used as a control). Additional cells were plated in 30 wells to serve as the day 1 (pretreatment) comparison. After 5 days of compound exposure, Cell Titer-Glo reagent was added to the cells and the relative luminescence units (RLUs) of each well was determined Cell Titer-Glo was also added to 32 μL of medium without cells to obtain a background value. The plates were allowed to incubate at room temperature for 10 minutes to stabilize luminescent signal and the luminescence signal was recorded with EnSpire. The relative increase in cell number of each sample is determined as follows: (RLU sample-RLU background/RLU estrogen only treated cells-RLU background)×100=% inhibition. The results are shown in Table 4.

### Example B

ER Degradation Determination by Western Blot

MCF-7 cells were plated at 0.3 million cells/mL (3 mL/well) in 6-well plates in experiment media and incubated at 37° C., 5% CO₂ for 48 hours. A 10× solution of compounds were made in DMSO and added the solution to the cells to achieve a final concentration of 10 μM. A DMSO control was included to enable a determination of the relative efficacy of test compounds. Fulvestrant was used as a positive control for ER alpha degradation, and 4-OH tamoxifen as a control for estrogen receptor stabilization. After incubating cells with the indicated compounds for 18-24 hours, cell lysates were prepared (2× Cell lysis buffer: 100 mM Tris, pH 8, 300 mM NaCl, 2% NP40, 1% Na deoxycholate, 0.04% SDS and 2 mM EDTA) and mixed thoroughly and incubated on ice. The protein concentration was quantified using BCA kit. Protein was separated on 4%-20% NuPAGE Novex 4-12% Bis-Tris Protein Gels using 1×MES running buffer. The gel was then transferred onto a nitrocellulose membrane. The blots were probed with antibodies against ESR1 protein (estrogen receptor alpha) (Santa Cruz, sc-8005). GAPDH protein was used as an internal control. The results are shown in Table 4.

### Example C

### ERα EC₅₀ Determination

MCF-7 cells were plated at 0.3 million cells/mL (3 mL/well) in 6-well plates in media (as described in Example A) and incubated at 37° C., 5% CO₂ for 48 hours. A 10 mM solution of compounds are made in DMSO and added the solution to the cells to achieve a final concentration of 10 μM. For EC₅₀ determination, MCF-7 cells were incubated with 3× or 5× serial dilutions of 10mM compounds, which provided final concentrations of the compounds from 10 μM to designed concentrations based on the potency of the compounds (for example, 0.0256 nM for the compound of Example 1). A DMSO control was included to enable a determination of the relative efficacy of test compounds. Fulvestrant was used as a positive control for ER alpha degradation, and 4-OH tamoxifen as a control for estrogen receptor stabilization. After incubating cells with compounds for 18-24 hours, cell lysates were prepared (2× Cell lysis buffer: 100 mM Tris, pH 8, 300 mM NaCl, 2% NP40, 1% Na deoxycholate, 0.04% SDS and 2 mM EDTA) and mixed thoroughly and incubated on ice. The protein concentration was quantified using BCA kit. Protein was separated on 4%-20% NuPAGE Novex 4-12% Bis-Tris Protein Gels using 1×MES running buffer. The gel was then transferred onto a nitrocellulose membrane and the blots were probed with antibodies against ESR1 protein (Santa Cruz, sc-8005) with GAPDH protein as an internal control. The blots were imaged on Azure C₆₀₀ Imager and the band density of the blots was quantified with Azurespot. EC₅₀ was calculated with GraphpadPrism. The results are shown in Table 4.

TABLE 4

| | MCF7 | ERα | ERα |
|---|---|---|---|
| Example | IC₅₀ (nM) | % degradation | EC₅₀ (nM) |
| fulvestrant | A | A | A |
| AZD9496 | A | A | A |
| ARN810 | A | A | A |
| Example 1 | A | B | A |

For MCF7 IC₅₀: A = a single IC₅₀ ≤ 25 nM; B = a single IC₅₀ ≥ 25 nM and ≤250 nM; C = a single IC₅₀ ≥ 250 nM. For ERα % degradation: A = ERα % degradation ≥ 80%; B = ERα % degradation < 80%. For ERα EC₅₀: A = a single EC₅₀ ≤ 25 nM; B = a single EC₅₀ ≥ 25 nM and ≤ 250 nM; C = a single EC₅₀ ≥ 250 nM.

### Example D

### Pharmacokinetic Determination

Grouping female CD mice weighing 20-30 g or female SD rats weighing 200-300 g randomly to two groups; one group was administered with test compound at a dose of 3.0 mg/kg by intravenous injection, the other group was administered with test compound at a dose of 10.0 mg/kg orally. The formulation for IV groups were DMSO/PEG400/150 mM glycine (pH 9) (5/10/85) and the formulation for PO groups were PEG400/PVP/Tween 80/0.5% CMC in water (9/0.5/0.5/90). After administering, blood samples of intravenous injection group were collected at time points of predose, 0.0833, 0.25, 0.5, 1, 2, 4, 8, 12 and 24 h; blood samples of oral group were collected at time points of predose, 0.25, 0.5, 1, 2, 4, 8, 12, and 24 h. Standard curve was plotted based on concentrations of the samples in a suitable range, the concentrations of test compounds in plasma samples were determined by using LC-MS/MS. Pharmacokinetic parameters were calculated according to drug concentration-time curve using a noncompartmental method by WinNonLin (Phoenix™, version 6.1) or other similar software.

TABLE 5
Mouse and Rat PK for Example 1

| | Dose | AUC_{inf} | Cl | V_{dss} | Cₘₐₓ | Tₘₐₓ | T_{1/2} | F |
|---|---|---|---|---|---|---|---|---|
| Species | method | (μM*h) | (mL · min⁻¹ · kg⁻¹) | (L/Kg) | (μM) | (h) | (h) | (%) |
| mouse | IV | 2.4 | 49.6 | 6.0 | — | — | 3.9 | — |
| | PO | 7.0 | — | — | 2.5 | 0.25 | 3.4 | 88 |
| rat | IV | 6.3 | 19 | 2.9 | — | — | 3.9 | — |
| | PO | 11.1 | — | — | 5.7 | 0.5 | 8.0 | 53 |

While the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound of Formula (I), or a pharmaceutically acceptable salt thereof, having the structure: 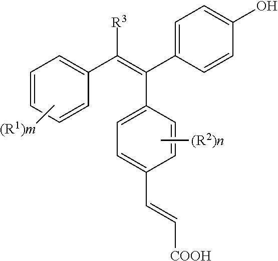
wherein:
one or more of R¹ is independently selected from the group consisting of halogen, —CN, an optionally substituted C₁-C₆ alkyl, an unsubstituted C₁-C₆ haloalkyl and an optionally substituted C₃-C₆ cycloalkyl;
one or more of R² is independently selected from the group consisting of halogen, —CN, an optionally substituted C₁-C₆ alkyl, an unsubstituted C₁-C₆ haloalkyl and an optionally substituted C₃-C₆ cycloalkyl;
R³ is selected from the group consisting of an optionally substituted C₁-C₆ alkyl, an unsubstituted C₁-C₆ haloalkyl and an optionally substituted C₃-C₆ cycloalkyl;
m is 0, 1, 2, 3, 4 or 5;
n is 0, 1, 2, 3 or 4; and
when m is 0, then n is 1, 2, 3 or 4.

2. The compound of claim 1, wherein m is 1.

3. The compound of claim 1, wherein m is 2.

4. The compound of claim 1, wherein m is 3.

5. The compound of claim 1, wherein m is 4.

6. The compound of claim 1, wherein m is 5.

7. The compound of any one of claims 1-6, wherein one or more of R¹ is independently halogen.

8. The compound of claim 7, wherein one or more of R¹ is fluoro.

9. The compound of claim 7, wherein one or more of R¹ is chloro.

10. The compound of any one of claims 1-9, wherein one or more of R¹ is —CN.

11. The compound of any one of claims 1-10, wherein one or more of R¹ is independently an optionally substituted C₁-C₆ alkyl.

12. The compound of claim 11, wherein one or more of R¹ is independently an unsubstituted C₁-C₆ alkyl.

13. The compound of claim 12, wherein one or more of R¹ is methyl.

14. The compound of any one of claims 1-13, wherein one or more of R¹ is independently an unsubstituted C₁-C₆ haloalkyl.

15. The compound of any one of claims 1-14, wherein one or more of R¹ is independently an optionally substituted C₃-C₆ cycloalkyl.

16. The compound of any one of claims 1-15, wherein n is 0.

17. The compound of any one of claims 1-15, wherein n is 1.

18. The compound of any one of claims 1-15, wherein n is 2.

19. The compound of any one of claims 1-15, wherein n is 3.

20. The compound of any one of claims 1-15, wherein n is 4.

21. The compound of any one of claims 1-15 and 17-20, wherein one or more of R² is independently halogen.

22. The compound of claim 21, wherein one or more of R² is fluoro.

23. The compound of claim 21, wherein one or more of R² is chloro.

24. The compound of any one of claims 1-15 and 17-23, wherein one or more of R² is —CN.

25. The compound of any one of claims 1-15 and 17-24, wherein one or more of R² is independently an optionally substituted C₁-C₆ alkyl.

26. The compound of claim 25, wherein one or more of R² is independently an unsubstituted C₁-C₆ alkyl.

27. The compound of claim 26, wherein one or more of R² is methyl.

28. The compound of any one of claims 1-15 and 17-27, wherein one or more of R² is independently an unsubstituted C₁-C₆ haloalkyl.

29. The compound of any one of claims 1-15 and 17-28, wherein one or more of R² is independently an optionally substituted C₃-C₆ cycloalkyl.

30. The compound of claim 1, wherein m is 1, 2 or 3; and one or more of R¹ is independently selected from the group consisting of halogen, an unsubstituted C₁-C₆ alkyl and an unsubstituted C₁-C₆ haloalkyl.

31. The compound of claim 30, wherein m is 1 or 2; and one or more of R¹ is independently halogen or an unsubstituted C₁-C₆ alkyl.

32. The compound of claim 30 or 31, wherein one or more of R¹ is fluoro.

33. The compound of claim 30 or 31, wherein one or more of R¹ is chloro.

34. The compound of claim 30 or 31, wherein one or more of R¹ is methyl.

35. The compound of any one of claims 30-34, wherein n is 0, 1 or 2; and one or more of R² is independently selected from the group consisting of halogen, an unsubstituted C₁-C₆ alkyl and an unsubstituted C₁-C₆ haloalkyl.

36. The compound of claim 35, wherein n is 0.

37. The compound of claim 35, wherein n is 1 or 2; and one or more of R² is independently halogen or an unsubstituted C₁-C₆ alkyl.

38. The compound of claim 35 or 37, wherein one or more of R² is fluoro.

39. The compound of claim 35 or 37, wherein one or more of R² is chloro.

40. The compound of claim 35 or 37, wherein one or more of R² is methyl.

41. The compound of any of claims 1-40, wherein R³ is an optionally substituted C₁-C₆ alkyl.

42. The compound of any of claims 1-41, wherein R³ is an optionally substituted C₁-C₃ alkyl.

43. The compound of any of claims 1-42, wherein R³ is an unsubstituted C₁-C₃ alkyl.

44. The compound of claim 43, wherein R³ is unsubstituted ethyl.

45. The compound of any of claims 1-40, wherein R³ is an unsubstituted C₁-C₆ haloalkyl.

46. The compound of claim 45, wherein R³ is an unsubstituted C₁-C₃ haloalkyl.

47. The compound of any of claims 1-40, wherein R³ is an optionally substituted C₃-C₆ cycloalkyl.

48. The compound of claim 47 wherein R³ is an unsubstituted C₃-C₆ cycloalkyl.

49. The compound of claim 1, wherein m is 2; n is 0; one R¹ is chloro; and the other R¹ is fluoro.

50. The compound of claim 1, wherein m is 2; n is 0; one R¹ is fluoro; and the other R¹ is unsubstituted methyl.

51. The compound of claim 1, wherein m is 1; n is 0; and R¹ is chloro.

52. The compound of claim 1, wherein m is 2; n is 2; one R¹ is chloro; the other R¹ is fluoro; and each R² is fluoro.

53. The compound of claim 1, wherein m is 2; n is 2; one R¹ is fluoro; the other R¹ is unsubstituted methyl; and each R² is fluoro.

54. The compound of claim 1, wherein m is 1; n is 2; R¹ is chloro; and each R² is fluoro.

55. The compound of claim 1, wherein R³ is unsubstituted ethyl, m is 2; n is 0;
one R¹ is chloro; and the other R¹ is fluoro.

56. The compound of claim 1, wherein R³ is unsubstituted ethyl, m is 2; n is 0;
one R¹ is fluoro; and the other R¹ is unsubstituted methyl.

57. The compound of claim 1, wherein R³ is unsubstituted ethyl, m is 1; n is 0;
and R¹ is chloro.

58. The compound of claim 1, wherein R³ is unsubstituted ethyl, m is 2; n is 2;
one R¹ is chloro; the other R¹ is fluoro; and each R² is fluoro.

59. The compound of claim 1, wherein R³ is unsubstituted ethyl, m is 2; n is 2;
one R¹ is fluoro; the other R¹ is unsubstituted methyl; and each R² is fluoro.

60. The compound of claim 1, wherein R³ is unsubstituted ethyl, m is 1; n is 2; R¹ is chloro; and each R² is fluoro.

61. The compound of claim 1 having a structure selected from the group consisting of: 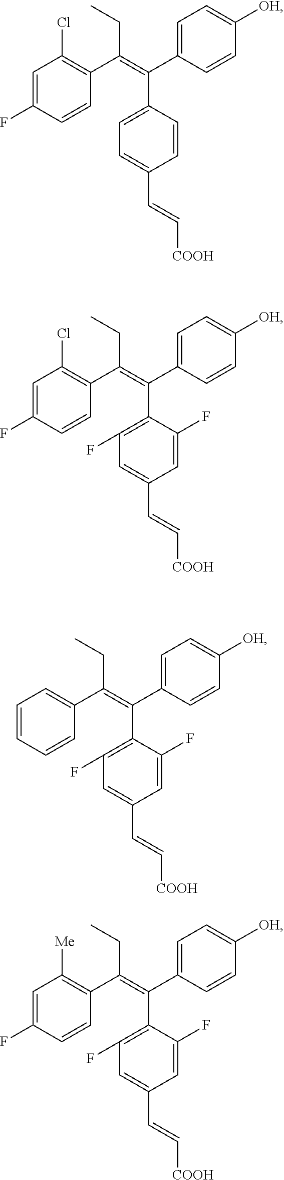 [SMILES: CC/C(=C(\c1ccc(O)cc1)c1ccc(/C=C/C(=O)O)cc1)c1ccc(F)cc1Cl ; CC/C(=C(\c1ccc(O)cc1)c1c(F)cc(/C=C/C(=O)O)cc1F)c1ccc(F)cc1Cl ; CC/C(=C(\c1ccc(O)cc1)c1c(F)cc(/C=C/C(=O)O)cc1F)c1ccccc1 ; CC/C(=C(\c1ccc(O)cc1)c1c(F)cc(/C=C/C(=O)O)cc1F)c1ccc(F)cc1C] 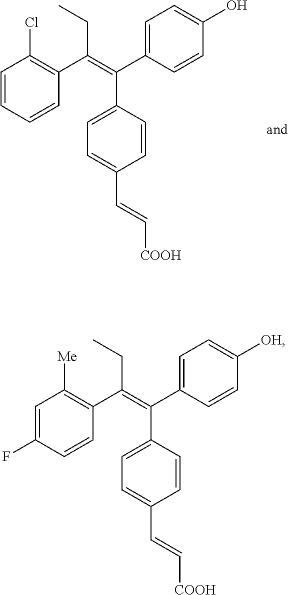 [SMILES: CC/C(=C(\c1ccc(O)cc1)c1ccc(/C=C/C(=O)O)cc1)c1ccccc1Cl ; CC/C(=C(\c1ccc(O)cc1)c1ccc(/C=C/C(=O)O)cc1)c1ccc(F)cc1C]
or a pharmaceutically acceptable salt of any of the foregoing.

62. A pharmaceutical composition comprising an effective amount of the compound of any one of claims 1-61, or a pharmaceutically acceptable salt thereof, and a pharmaceutically acceptable carrier, diluent, excipient or combination thereof.

63. Use of a compound of any one of claims 1-61, or a pharmaceutically acceptable salt thereof, or the pharmaceutical composition of claim 62 in preparation of a medicament for the treatment of a disease or condition that is ER alpha dependent and/or ER alpha mediated.

64. The use of claim 63, wherein the disease or condition is selected from the group consisting of a lung cancer, a breast cancer and a gynecological cancer.

65. The use of claim 63, wherein the disease or condition is selected from the group consisting of a lung cancer, a breast cancer, an endometrial cancer, an ovarian cancer and a cervical cancer.

66. The use of claim 63, wherein the disease or condition is a breast cancer.

67. The use of claim 63, wherein the disease or condition is a lung cancer.

68. The use of claim 63, wherein the disease or condition is a gynecological cancer.
